(19) United States
(12) Patent Application Publication
(10) Pub. No.: US 2024/0139739 A1
(43) Pub. Date: May 2, 2024

(54) A MICROFLUIDIC CHANNEL INCLUDING A FLUIDIC PUMP TO DIRECT A CELL THROUGH A CONSTRICTION REGION

(71) Applicant: Hewlett-Packard Development Company, L.P., Spring, TX (US)

(72) Inventors: Milo OVERBAY, Corvallis, OR (US); Viktor SHKOLNIKOV, Palo Alto, CA (US); Roberto A. PUGLIESE, Corvallis, OR (US)

(73) Assignee: Hewlett-Packard Development Company, L.P., Spring, TX (US)

(21) Appl. No.: 18/278,365

(22) PCT Filed: Feb. 26, 2021
(86) PCT No.: PCT/US2021/019946

§ 371 Date: Aug. 22, 2023

(51) Int. Cl. B01L 3/00

(52) U.S. Cl. CPC B01L 3/502746; B01L 3/502715; B01L 2200/0663; B01L 2400/086

(57) ABSTRACT

An example apparatus comprises includes a first reservoir to store a biologic sample containing a cell, a microfluidic channel fluidically coupled to the first reservoir, and circuitry. The microfluidic channel includes a constriction region including a first circumference that is attenuated from remaining portions of the microfluidic channel, and a fluidic pump disposed within the microfluidic channel. The circuitry is to activate the fluidic pump to direct flow of the cell from the first reservoir to the microfluidic channel and through the constriction region.

### BACKGROUND

Cell transfection is a process of introducing transfection material, such as nucleic acids, proteins, or other molecules or particles, inside a cell. Cell transfection may be used for a variety of different applications, including but not limited to gene therapy, gene editing, and pharmaceutical applications.

### BRIEF DESCRIPTION OF THE DRAWINGS

FIGS. 1A-1B illustrate an example apparatus including a first reservoir, a microfluidic channel including a fluidic pump, and circuitry, in accordance with the present disclosure.

FIG. 2 illustrates an example method of transfecting a cell using a fluidic pump disposed within a microfluidic channel, in accordance with the present disclosure.

FIG. 3 illustrates another example apparatus including a first reservoir, a first microfluidic channel, a second microfluidic channel, and a fluidic pump, in accordance with the present disclosure.

FIGS. 4A-4H illustrate a variety of different example apparatuses for transfecting cells, in accordance with the present disclosure.

FIGS. 5A-5D illustrate a variety of different example apparatuses for transfecting cells and which include multiple microfluidic channels, in accordance with the present disclosure.

FIGS. 6A-6B illustrates an example apparatus for transfecting cells and which includes a micromixer, in accordance with the present disclosure.

FIGS. 7A-7F illustrate a variety of different example apparatuses for transfecting cells and which include recirculation loops, in accordance with the present disclosure.

FIG. 8 illustrates an example apparatus for performing cell transfection using a recirculation loop, in accordance with the present disclosure.

### DETAILED DESCRIPTION

In the following detailed description, reference is made to the accompanying drawings which form a part hereof, and in which is shown by way of illustration of specific examples in which the disclosure may be practiced. It is to be understood that other examples may be utilized, and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

Biological cells are the basic building blocks of skin, tissues, and other materials. Cells and their organelles are enveloped by thin membranes that separate their chemical contents from the extracellular environment. As noted above, cell transfection refers to the process of introducing transfection material, such as nucleic acids, proteins, or other molecules or particles, inside a cell. Transfecting cells may be performed using viral transfection, lipofection, and electrotransfection techniques. Viral transfection is laborious and results in the introduction of viral components into the cell, which may be unwanted for various applications. Viral transfection additionally is limited in the size of transfection material that may be moved into the intracellular space, which inhibits transfecting transfection material such as Cluster regularly interspaced short palindromic repeats (CRISPR) associated protein 9 (Cas9) protein for CRISPR, quantum dots, deoxyribonucleic acid (DNA) or ribonucleic acid (RNA) above a threshold size, peptides, proteins, antibodies and nanoparticles. Lipofection is also laborious and introduces surfactants into the cell or cell membrane. Electrotransfection is a manual process that places cells in a transfection chamber and removes the cells, one batch at a time, making it difficult to explore transfection conditions and/or to transfect at the single cell level. Examples in accordance with the present disclosure are directed to mechanically transfecting cells using a fluidic pump disposed within a microfluidic channel of the apparatus. The mechanical transfection may be applied at a single cell level, at multiple cell levels using parallel processing, and/or used to assess the transfection conditions for transfecting the cells and/or to supply multiple transfection materials per run.

Examples in accordance with the present disclosure are directed to apparatuses that perform mechanical cell transfection, such as a compact, integrated microfluidic apparatus. The biologic sample is introduced to the apparatus, which includes fluidic pumps to actively move fluid through microfluidic channels of the apparatus. A fluidic pump of the apparatus may be actuated to cause a cell of the biologic sample to flow into and through a constriction region of the microfluidic channel. The constriction region has a circumference that is attenuated from remaining portions of the microfluidic channel, thereby increasing flow velocity through the constriction region from the remaining portions. As used herein, a circumference of the constriction region or other portions of the microfluidic channel includes and/or refers to a distance around a perimeter of the constriction region or portion of the microfluidic channel. A circumference that is attenuated, as used herein, includes and/or refers to a reduced circumference as compared to the circumference of the remaining portions of the microfluidic channel. In some examples, the constriction region with the attenuated circumference may have a hydraulic diameter that is smaller than the hydraulic diameter of the remaining portions of the microfluidic channel. In some examples, the hydraulic diameter of the constriction region may be smaller than the nominal diameter of the cell. In some examples, a construction region may be a portion of the microfluidic channel that has an oblong cross-sectional shape, such that a diameter along the width is different than a diameter along the height of the microfluidic channel. In some examples, a transection of the circumference of the constriction region may be smaller than the nominal diameter of the cell such that the cell is deformed when travelling through the constriction region. A transection of the circumference includes and/or refers to a cross-section or a slice of the constriction region taken at a point along the length of the constriction region. The flow of the cell through the constriction region may cause the cell to form apertures in the cell membrane sufficient for transfection material to transition through.

For example, the flow rate of the cell may be used to apply a set shear force to cause the apertures in the cell membrane to form, which are sometimes referred to as “pores”. As used herein, a shear force includes and/or refers to a force caused by parallel forces acting on the cell in opposite directions and at different parts of the cell. For example, the cell may contact a wall of the constriction region while flowing through the constriction region, which may cause a pulling force on the cell in the direction of the fluid flow and a pulling force on the cell in the opposite direction of the fluid flow. The shear force applied may be set to cause apertures of a sufficient size for a particular transfection material to pass through. Too much shear force may result in cell lysing or death, and too little shear force may result in no apertures or apertures of an insufficient size for transfection. The mechanical transfection may not produce contaminates, may be used to transfect at the single cell level, may be used to transfect multiple types of cells and/or transfection material in one microfluidic device, and/or to optimize transfection conditions for a particular type of cell and particular type of transfection material.

In a particular example, an apparatus may comprise a first reservoir, a microfluidic channel fluidically coupled to the first reservoir, and circuitry. The first reservoir is to store a biologic sample containing a cell. The microfluidic channel includes a constriction region and a fluidic pump disposed within the microfluidic channel. The constriction region includes a first circumference that is attenuated from remaining portions of the microfluidic channel. The circuitry is to activate the fluidic pump to direct flow of the cell from the first reservoir into the microfluidic channel and through the constriction region. In some examples, the flow rate may be set based on the particular type of cell. For example, a particular shear force to be applied to the cell may be set based on the type of cell, and may be dependent on the flow rate and dimensions of the constriction region.

As another example, a method for performing cell transfection may include receiving, at a microfluidic channel, a biologic sample containing a cell and transfection material. The method includes forming apertures in the cell membrane of the cell by directing a flow of the cell through a constriction region of the microfluidic channel, wherein the flow of the cell is directed using a fluidic pump disposed within the microfluidic channel, and wherein the constriction region includes a first circumference that is attenuated from remaining portions of the microfluidic channel. The method further includes transfecting the cell by exposing the cell with the apertures to the transfection material.

As a further example, an apparatus may comprise a first reservoir to store a biologic sample containing a plurality of cells, a first microfluidic channel fluidically coupled to the first reservoir, a second microfluidic channel fluidically coupled to the first reservoir, and a fluidic pump. The first microfluidic channel includes a first constriction region that includes a first circumference that is attenuated from remaining portions of the first microfluidic channel. The second microfluidic channel includes a second constriction region that includes a second circumference that is attenuated from remaining portions of the second microfluidic channel. The first constriction region and second constriction region may include different dimensions from one another, which may be used to provide different shear forces on cells of the plurality of cells which are directed through the respective constriction regions. The fluidic pump is in fluidic communication with the first microfluidic channel and with the second microfluidic channel to cause movement of respective portions of the biologic sample from the first reservoir to the first microfluidic channel, and to the second microfluidic channel and respectively through the first constriction region and the second constriction region.

The shear force applied to the cells may be dependent on the flow rate and dimensions of the respective constriction regions. In some examples, the flow rate may remain the same between the first and second constriction regions to apply different shear forces on respective cells. In some examples, the flow rate may be adjusted between the first and second constriction regions to apply a set shear force on the respective cells. In some examples, the plurality of cells may include different types of cells, and the flow rates may be adjusted to apply respective set shear forces to the different types of cells.

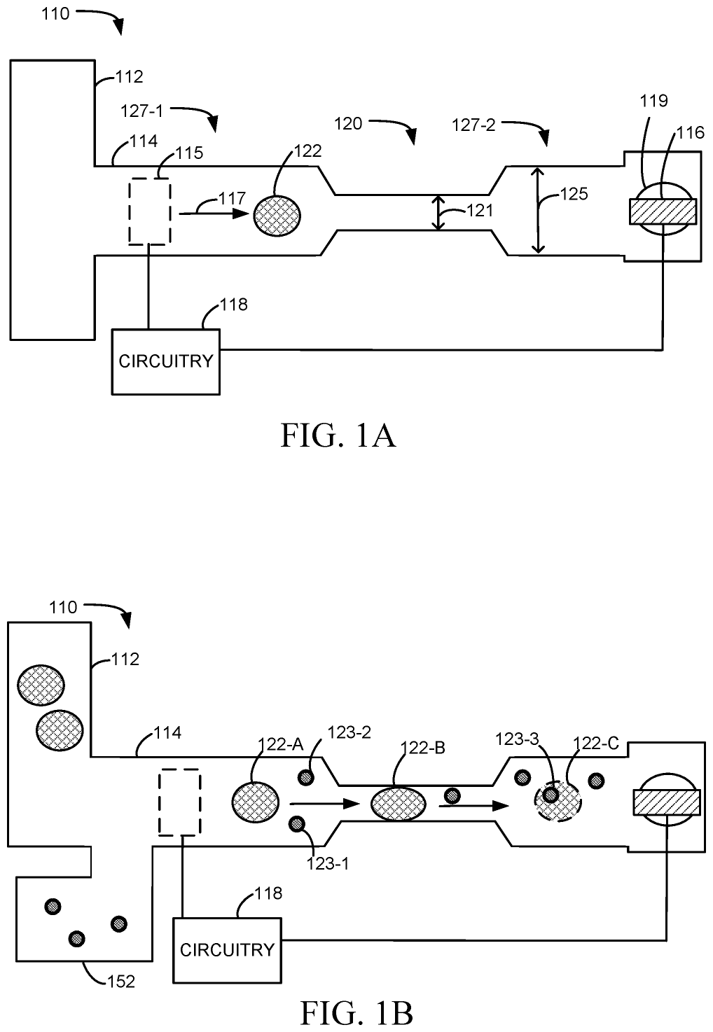

Turning now to the figures, FIGS. 1A-1B illustrate an example apparatus including a first reservoir, a microfluidic channel including a fluidic pump, and circuitry, in accordance with the present disclosure. As shown by FIG. 1A, the apparatus 110 includes a first reservoir 112 to store a biologic sample containing a cell 122, a microfluidic channel 114 fluidically coupled to the first reservoir 112, and circuitry 118.

In various examples, the apparatus 110 may include a microfluidic device and the coupled circuitry 118 that controls components of the microfluidic device. The microfluidic device may include the first reservoir 112, the microfluidic channel 114, a constriction region 120, and a fluidic pump, such as the ejection nozzle, as further described below. In some examples, the circuitry 118 forms part of the microfluidic device.

The microfluidic channel 114 may include a constriction region 120 and the fluidic pump disposed within the microfluidic channel 114. The constriction region 120 includes a first circumference 121 that is attenuated from remaining portions 127-1, 127-2 of the microfluidic channel 114, as illustrated by the second circumference 125 of the microfluidic channel 114 which is larger than the first circumference 121. Although the arrows showing the first circumference 121 and the second circumference 125 are illustrated as linear, the first circumference 121 and the second circumference 125 are along the perimeter of the constriction region 120 and the remaining portions 127-1, 127-2 of the microfluidic channel 114.

The microfluidic channel 114 may pass a portion of a biologic sample, which may include a cell 122. The biologic sample may be passed via the fluidic pump that actively moves fluid through the microfluidic channel 114, as shown by arrow 117 illustrating fluid flow.

In some examples, the biologic sample may be mixed with a medium and/or a buffer, and loaded into the first reservoir 112 that is coupled to the microfluidic channel 114. Example transfection material includes nucleic acids, such as DNA or RNA, proteins, and other molecules or particles. For example, the transfection material may include guide RNA, Cas9 protein for CRISPR, and/or other enzymes used for gene editing.

Fluid flow may be caused by the actuation of the fluidic pump that is internal to the microfluidic channel 114. For example, the fluidic pump may be fired or pulsed, which creates the fluid flow by pushing or pulling fluid within the microfluidic channel 114. As further described below, in some examples, the fluidic pump includes a thermal inkjet (TIJ) resistor. Activation of the TIJ resistor may create the flow of fluid by firing drops of fluid from the microfluidic channel 114 and/or creating a vapor bubble. As a specific example, the resistor 116 may be actuated to cause firing of drops of fluid from an ejection nozzle, which creates the fluid flow through the microfluidic channel 114 by pulling the fluid. The cell 122 may be carried along the microfluidic channel 114 by the fluid flow in response to firing or pulsing the resistor 116, with the number of fired drops to move the cell 122 through the microfluidic channel 114 to the ejection nozzle varying depending on the flow path. In some examples, the fluidic pump includes another resistor, such as resistor 115, that may be fired or pulsed to cause a vapor bubble within the microfluidic channel 114 and that creates the fluid flow. The fluid flow causes the cell 122 to travel along the microfluidic channel 114 into and through the constriction region 120 and out of the constriction region 120. In some examples, the flow into, through, and/or out of the constriction region 120 may cause application of shear force on the cell 122 due to the first circumference 121 of the constriction region 120 and/or the change between the first circumference 121 of the constriction region 120 and the circumference of the remaining portions 127-1, 127-2 of the microfluidic channel 114, as described further below. The flow rate may be dependent on a firing or pulse rate of resistor 116 and/or resistor 115, which may be adjusted to set the flow rate and set the shear force applied to the cell 122.

In some examples, the fluidic pump may include an inertial pump that actuates the fluid. In some examples, the fluidic pump may include a resistor, such as a thermal resistor. An example thermal resistor includes a TIJ resistor. However, examples are not so limited and a variety of different types of resistors may be used. Other example fluidic pumps include an integrated inertial pump, a piezoelectric device, a magnetostrictive element, an ultrasound source, and other suitable pumps. In some examples, and as illustrated by FIG. 1A, the fluidic pump includes an ejection nozzle which is located at an opposite end of the microfluidic channel 114 from the first reservoir 112. The ejection nozzle may be used for ejecting fluid from the microfluidic channel 114.

In some examples, the ejection nozzle may include a resistor 116 and an orifice 119 located near the resistor 116. The orifice 119 may be used for ejecting fluid from the microfluidic channel 114, such as ejecting fluid from the microfluidic device. In some examples, the orifice 119 may include an interface to another microfluidic channel or a chamber of the microfluidic device for further processing and/or analysis. In some examples, the resistor 116 may be first activated to direct the flow of the cell 122 along the microfluidic channel 114 and activated a second time to eject the cell 122 from the microfluidic channel 114, such as in the direction of the arrow 117. For example, the resistor 116 may act as the fluidic pump to pull fluid including the cell 122 through the microfluidic channel 114 and eject fluid from the orifice 119. The cell 122 may be ejected onto a new fluid path and/or out of the microfluidic device onto a substrate. In some examples, the orifice 119 may be defined by a surface of the microfluidic channel 114, through which fluid may be ejected in drops for further processing.

However, examples are not so limited. In some examples, the fluidic pump may include a resistor 115 disposed within the microfluidic channel 114 which does not form part of ejection nozzle and which may be used to push and/or pull fluid within the microfluidic channel 114. The resistor 115 may be located on an end of the microfluidic channel 114 that is proximal to the first reservoir 120 and the resistor 115 may push the fluid in the direction of the arrow 117. In some examples, although not illustrated, the resistor 115 may be located on the opposite end of the microfluidic channel 114 from the first reservoir 112 and upstream from the ejection nozzle including the resistor 116, and may pull the fluid in the direction of the arrow 117. In some examples, the apparatus 110 may include the resistor 115 and the ejection nozzle disposed within the microfluidic channel 114. As further described herein, the circuitry 118 may activate the resistor 116 of the ejection nozzle to eject the cell 122 to a chamber, where the chamber includes transfection material and, in response, exposes the cell 122 to the transfection material.

The circuitry 118 may activate the fluidic pump, such as the resistor 116 of the ejection nozzle, to direct the flow of the cell 122 from the first reservoir 112 into the microfluidic channel 114 and through the constriction region 120. As further illustrated by FIG. 1B and described below, the flow of the cell 122 through the constriction region 122 may cause application of shear force on the cell 122, which may cause formation of apertures in the cell membrane of the cell 122. In some examples, the first circumference 121 of the constriction region 120 may constrict the cell 122 from ninety percent to thirty percent of a nominal diameter of the cell 122, however examples are not so limited.

For example, the circuitry 118 may activate the fluidic pump to direct flow of the cell 122 to apply shear force on the cell 122 in response to the flow through the constriction region 120, and thereby cause formation of apertures in the cell membrane of the cell 122. In some examples, compression, tensile, and shear forces are applied on the cell 122. In some examples, the circuitry 118 may expose the cell 122 to transfection material to transfect the cell 122. As used herein, a compression force includes and/or refers to a force caused by opposing forces that push one part of the cell 122 in a first direction and push another part of the cell in a second and opposite direction. For example, a compression force may squeeze or decrease a length of the cell 122, and may be caused by the cell 122 entering the constriction region 120. A tensile force includes and/or refers to a force caused by opposing forces that pull one part of the cell 122 in a first direction and pull another part of the cell 122 in a second and opposite direction. For example, the tensile force may stretch or lengthen the cell 122, and may be caused by the cell 122 exiting the constriction region 120. As described above, a shear force may be applied to the cell 122 due to parallel forces acting on the cell 122 from the cell 122 contacting the wall of the constriction region 120 while being flown through the constriction region 120, which causes shear tension due to the parallel forces in the direction of the flow and opposite to the direction of the flow.

In some examples, when the cell 122 passes through the constriction region 120 and the remaining portions 127-1, 127-2 of the microfluidic channel 114, compression, tensile, and/or shear forces may be applied to the cell 122 which cause the formation of the apertures in the cell membrane. The amount of forces applied may be adjusted based on the initial flow rate of the fluid, a degree of attenuation, and dimensions of the constriction region 120 and the remaining portions 127-1,127-2 of the microfluidic channel 114 including circumferences and/or lengths. For example, the remaining portions 127-1, 127-2 of the microfluidic channel 114 may be respectively upstream and downstream from the constriction region 120 and may include the second circumference 125 that is larger than the first circumference 121 of the constriction region 120. The size and length of the remaining portions 127-1, 127-2 may impact a degree and duration of forces from acceleration and/or deceleration as the cell 122 enters into and/or exits from the constriction region 120 of the microfluidic channel 114. The first circumference 121 may impact a maximum flow rate and friction forces between the walls of the constriction region 120 and the cell membrane, and the length may impact the amount of time that the compression force, tensile force, and shear force is applied to the cell 122. The degree of attenuation may include and/or refer to a change in circumference between the constriction region 120 and the remaining portions 127-1, 127-2 and/or within the constriction region 120, such as a taper of circumference transitions between the constriction region 120 and the remaining portions 127-1, 127-2 and which may cause changes in forces applied on the cell 122 and/or changes in the flow rate when the cell 122 is flowing within the constriction region 120 and between or within the remaining portions 127-1, 127-2 of the microfluidic channel 114.

In some examples, the circuitry 118 activates the fluidic pump to direct the flow of the cell 122 into the microfluidic channel 114 and through the constriction region 120 at a set flow rate. As further described herein, the set flow rate may be specific to the type of cell and an amount of shear force to apply to cause apertures in the type of cell and for a type of transfection material. The flow rate may impact the amount of shear force applied on the cell 122, for example, by changing an amount of time that the cell 122 is exposed to the shear force when traveling through the constriction region 120. In some examples, as described further herein, the circuitry 118 may store a list that identifies the set flow rate for the particular type of cell and/or may generate the list.

The apparatus 110 may include a variety of variations, such as the use of different types of fluidic pumps, different arrangements of the fluidic pump and/or number of fluidic pumps, and/or additional microfluidic channels.

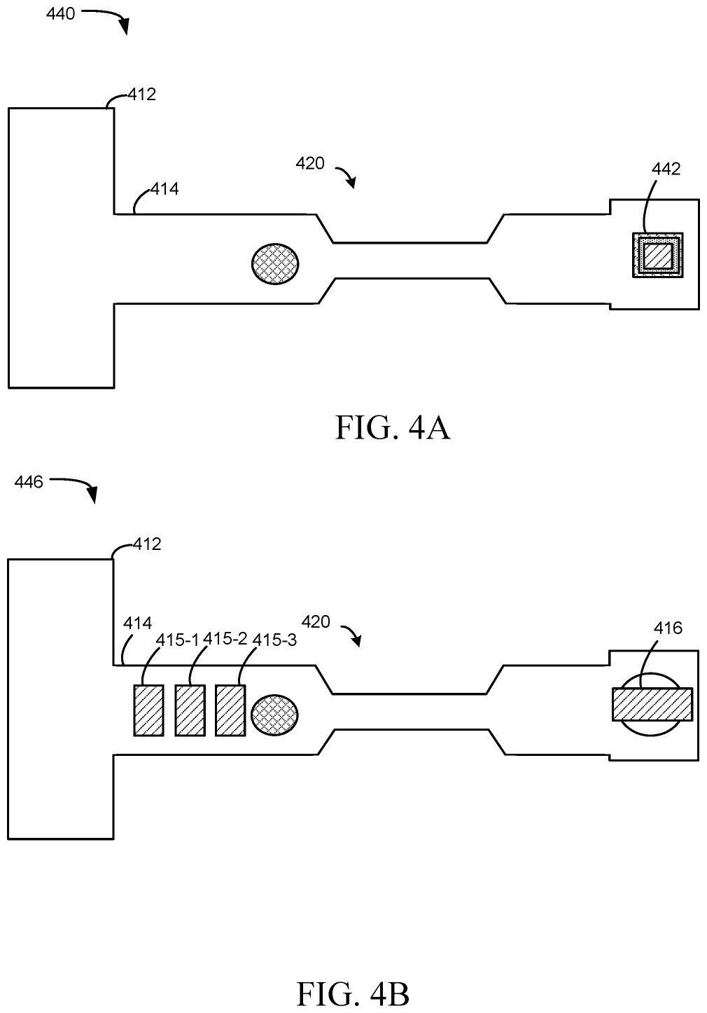

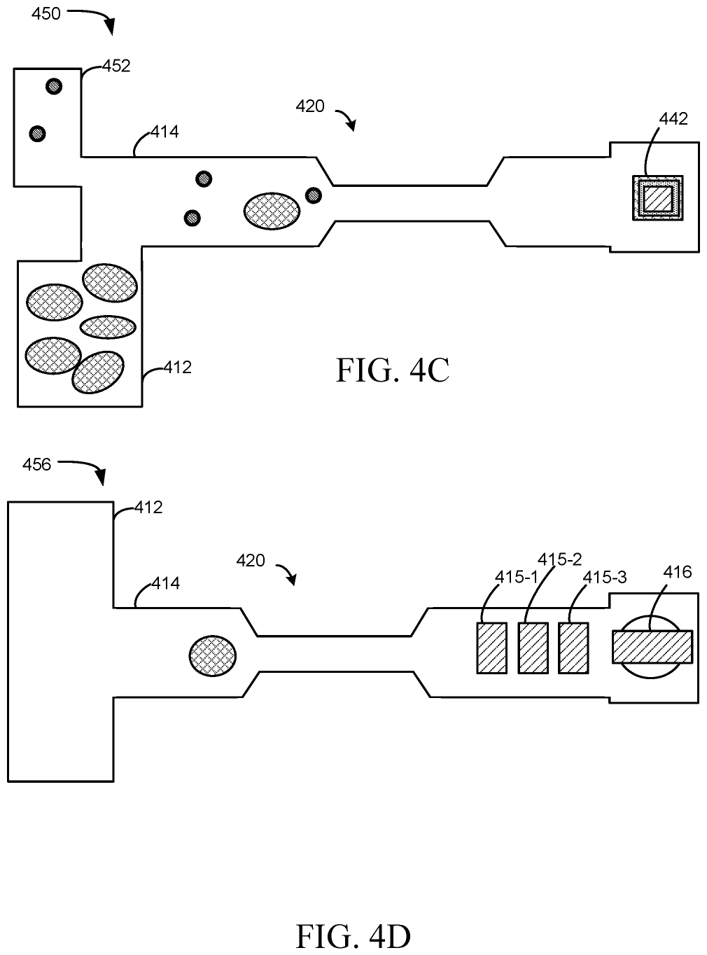

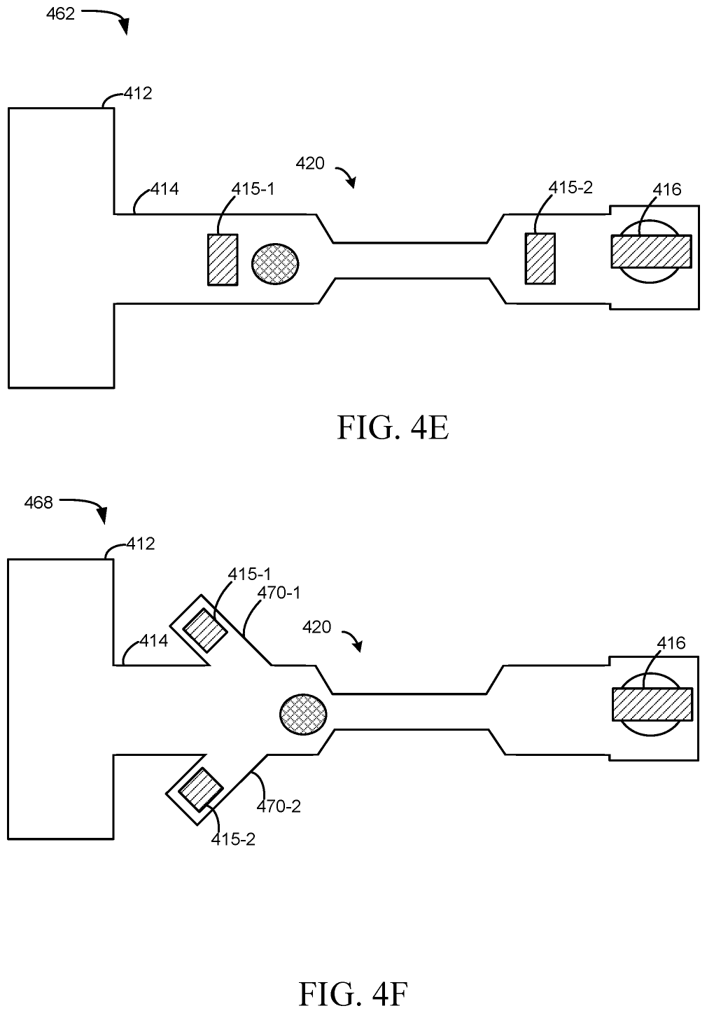

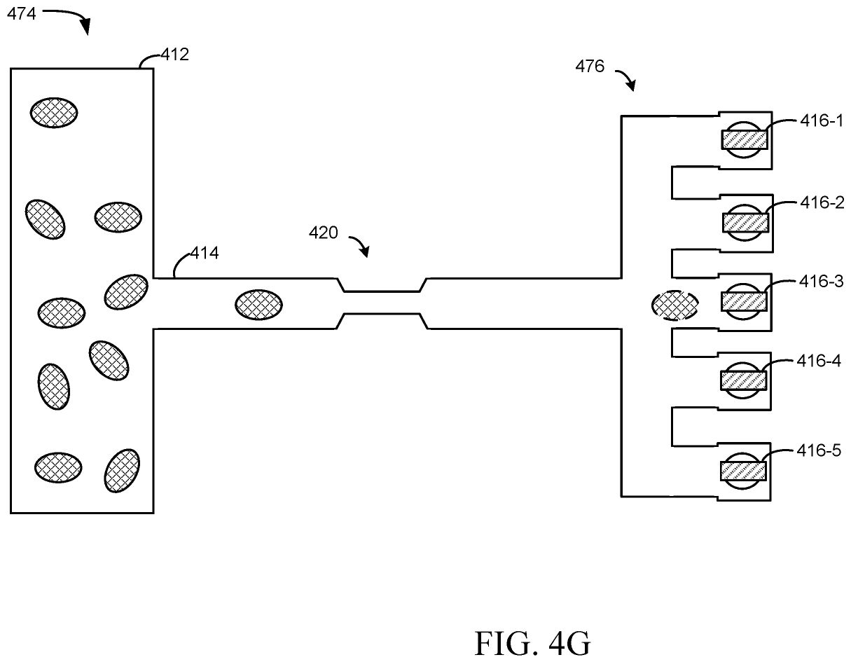

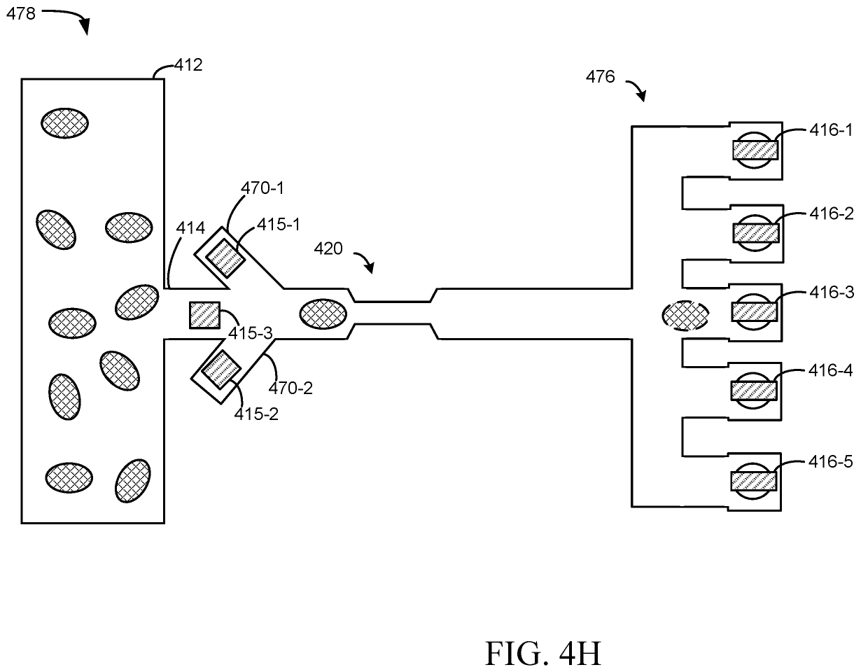

In some examples, as further illustrated by the channels 414, 470-1, 470-2 of at least FIG. 4F, the microfluidic channel 114 may include multiple channels. For example, the microfluidic channel 114 may include a first channel that includes the constriction region 120 and a second channel that intersects the first channel and is in fluidic communication with the first channel. The fluidic pump may be disposed within the second channel.

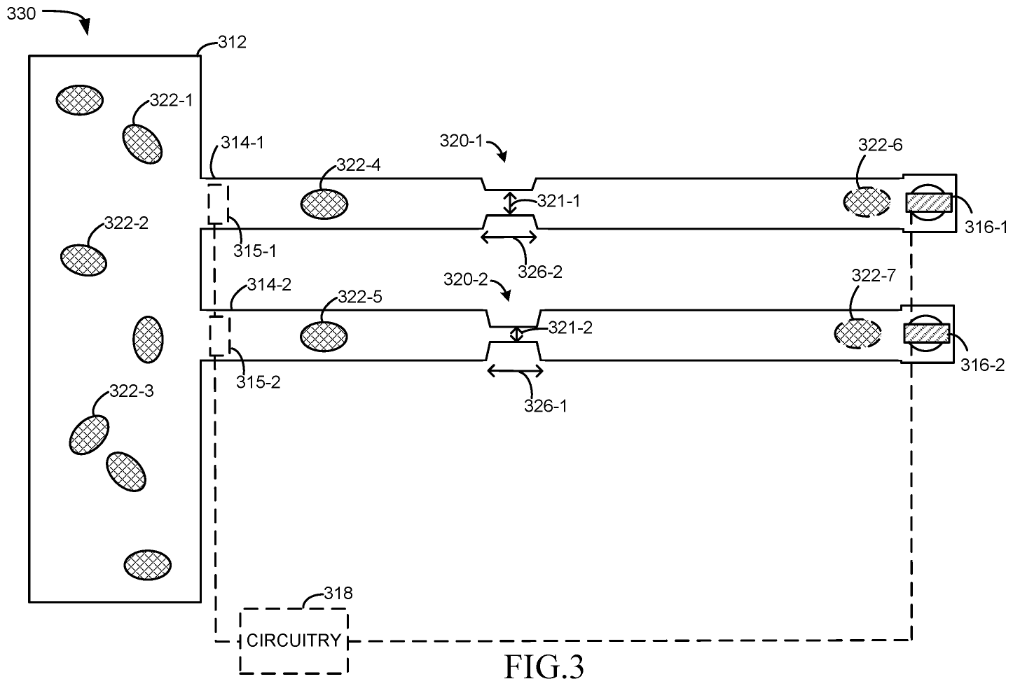

In some examples, the microfluidic channel 114 may include a first channel including the constriction region 120 and a second channel including a second constriction region, as further illustrated by the first microfluidic channel 314-1 and second microfluidic channel 314-2 of FIG. 3. The second constriction region may include a second circumference that is attenuated from remaining portions of the microfluidic channel 114, and wherein the first constriction region 120 and the second constriction region include different dimensions from one another. The circuitry 118 may activate the fluidic pump to direct the flow of the cell 122 from the first reservoir 112 to the first channel and through the constriction region 120, and direct flow of a second cell from the first reservoir 112 to the second channel and through the second constriction region. In some examples, the flow of fluid through the first channel and second channel may be at a set flow rate; however examples are not so limited.

In some examples, the fluidic pump includes a plurality of resistors disposed within the microfluidic channel 114. For example, the plurality of resistors may be arranged in series or may include resistors of different sizes and/or strengths which are co-located and/or stacked upon another in the microfluidic channel 114. In some examples, the circuitry 118 is to selectively activate the plurality of resistors to provide a first flow rate to flow the cell 122 to the microfluidic channel 114 (from the first reservoir 112) and provide a second flow rate to flow the cell 122 into and through the constriction region 120, wherein the second flow rate includes a change in velocity from the first flow rate. To provide different flow rates, the circuitry 118 may activate a resistor differently, such as for longer or shorter periods of time, and/or may activate different numbers of resistors to change the velocity. However, examples are not so limited.

In some examples, the apparatus 110 may include a second reservoir 152 fluidically coupled to the microfluidic channel 114, such as illustrated by FIG. 1B and FIG. 4C. The second reservoir 152 may store the transfection material. For example, the circuitry 118 may activate the fluidic pump to direct the flow of the cell 122 from the first reservoir 112 into the microfluidic channel 114 and through the constriction region 120, and to direct flow of the transfection material from the second reservoir 152 into the microfluidic channel 114 and through the constriction region 120 to transfect the cell 122.

In some examples, the first reservoir 112 stores the biologic sample and the transfection material, and the circuitry 118 is to activate the fluidic pump to direct the flow of the cell 122 and the transfection material from the first reservoir 112 into the microfluidic channel 114 and through the constriction region 120. In some examples, the second reservoir may be fluidically coupled to an output of the microfluidic channel 114, and used to allow the cell 122 to aggregate with other transfected cells, incubate, and/or recover.

In some examples, the apparatus 110 may further include a micromixer to mix the cell 122 with the transfection material. The micromixer may include a channel and/or chamber fluidically coupled to the microfluidic channel 114 and a second reservoir that stores the transfection material, as further illustrated herein. As used herein, a micromixer includes and/or refers to microparts, such as a channel, chamber and/or other parts, used to mix fluids. In some examples, the micromixer includes or is in fluidic communication with an energy source to drive the flow of fluid throughout other microparts and cause mixing of different fluids, such as fluid containing the cell 122 and fluid containing the transfection material. Example energy sources include a fluidic pump, as previously described, an electrode array or other source of an electrical field, an acoustic source, a magnet, a rotating plate or other source of centrifugal forces, among other energy sources. In some examples, the fluid may be mixed within the micromixer using passive mixing, such as using unbalanced collision channels, an embedded barrier channel, a convergent-divergent channel, a chamber with different channels, a three-dimensional spiral, an overbridge, among other types of passive micromixers.

FIG. 1B illustrate example operations of the apparatus 110 illustrated by FIG. 1A. As shown by FIG. 1B, the resistor 116 of the ejection nozzle may be activated by the circuitry 118 to direct the flow of the cell 122 from the first reservoir 112 to the microfluidic channel 114, as shown by the cell 122 at position 122-A, into and through the constriction region 120, as shown by the cell 122 at position 122-B, and out of the constriction region 120, as shown by the cell 122 at position 122-C. The resistor 116 may agitate a volume of fluid, as shown by the arrows, to move the cell 122 along the microfluidic channel 114.

When the cell 122 is directed into the constriction region 120, the cell 122 may be constricted, as shown by position 122-B, which causes the formation of the apertures, as illustrated by the dashed lines shown by position 122-C. In some examples, transfection material 123-1, 123-2, 123-3 is also directed through the microfluidic channel 114 and which exposes the cell 122 to the transfection material 123-1, 123-2, 123-3. Transfection material may pass through the apertures in the cell membrane and into the intracellular space of the cell 122, as shown by the cell 122 at position 122-C and the particular transfection material 123-3.

Although the above describes transfecting a cell, examples are not so limited. In various examples, a plurality of cells may be transfected at the same time and/or using multiple processes along the microfluidic channel 114. For example, the first reservoir 112 may store a biologic sample containing a plurality of cells.

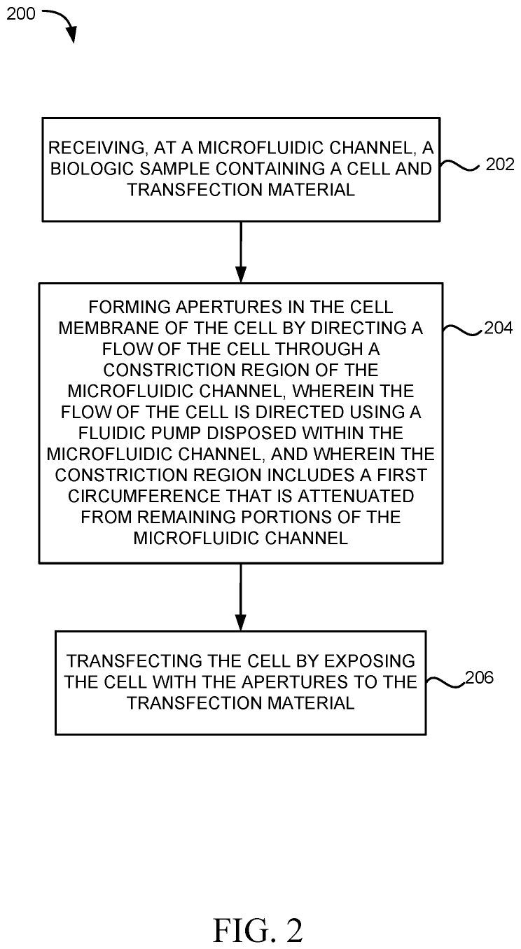

FIG. 2 illustrates an example method of transfecting a cell using a fluidic pump disposed within a microfluidic channel, in accordance with the present disclosure. The method 200 may be performed by the apparatus 110 of FIG. 1A and/or the apparatus 330 of FIG. 3, as further described herein.

At 202, the method 200 includes receiving, at a microfluidic channel, a biologic sample containing a cell and transfection material. The microfluidic channel may form part of a microfluidic device used to process and/or analyze the biologic sample.

In some examples, the biologic sample and transfection material may be mixed together prior to being received at the microfluidic channel. For example, the biologic sample and transfection material may be pre-mixed prior to loading into a first reservoir of a microfluidic device. In some examples, the biologic sample may be loaded into a first reservoir, the transfection material loaded into a second reservoir, and the method 200 may include mixing portions of the biologic sample and the transfection material prior to or after applying shear force to the cell, as further described herein.

At 204, the method 200 includes forming apertures in the cell membrane of the cell by directing a flow of the cell through a constriction region of the microfluidic channel. The flow of the cell is directed using a fluidic pump disposed within the microfluidic channel, and the constriction region includes a first circumference that is attenuated from remaining portions of the microfluidic channel. The change in circumferences between the constriction region and remaining portions of the microfluidic channel may impart acceleration and/or deceleration, and create compression, tensile, and shear forces on the cell. Additional forces may also occur. For example, a transection of the first circumference of the constriction region may be smaller than a nominal diameter of the cell. In some examples, directing the flow of the cell through the constriction region includes constricting the cell in a dimension from between ninety percent to thirty percent of a nominal circumference of the cell, and causing deformation of the cell and shear friction between the cell and walls of the constriction region while flowing the cell through the constriction region.

As previously described, when the cell passes through the constriction region and the remaining portions of the microfluidic channel, compression, tensile, and/or shear forces may be applied to the cell which cause the formation of the apertures in the cell membrane. In some examples, directing the flow of the cell through the constriction region may include applying compression, tensile, and shear forces on the cell by directing the flow of the cell into and through the constriction region, and removing the compression, tensile, and shear forces on the cell by directing the flow of the cell out of the constriction region. In some examples, the method 200 may further include incubating the cell with the transfection material to allow the transfection material to pass through the apertures and the apertures to close, thereby trapping the transfection material in the cell.

In some examples, the fluidic pump may be activated a plurality of times to direct the flow. For example, the fluidic pump may be activated a first time to direct flow of a portion of the biologic sample from the first reservoir to the microfluidic channel, and activated a second time to direct the flow of the portion of the biologic sample through the constriction region. In some examples, directing the flow of the cell through the constriction region includes changing a flow of a portion of the biologic sample from a first velocity to a second velocity, thereby flowing the cell into the constriction region and constricting the cell, and causing application of a set shear force on the cell.

For example, the fluidic pump may be fired or pulsed a plurality of times. Each pulse of the fluidic pump may move a volume of fluid including five to one hundred fifty picoliters (pL), and the fluidic pump may be fired at speeds of up to 10,000 to 50,000 hertz. The volume of fluid within the constriction region may be one to ten pL, such that one pulse of the fluidic pump may transport the cell through the constriction region. However, examples are not so limited.

In some examples, the fluidic pump is activated one time, and which directs the flow of the portion of the biologic sample from the first reservoir to the microfluidic channel and through the constriction region. The flow rate may be set to provide a set shear force for a particular type of cell when the cell travels through the constriction region.

At 206, the method 200 includes transfecting the cell by exposing the cell with the apertures to the transfection material. The cell with the apertures may be incubated with the transfection material and allowed to recover from the shear force applied. As described above, the transfection material may be smaller in circumference than the apertures, and may pass through the apertures of the cell membrane into the intracellular space. As the cell recovers, the apertures close and trap the transfection material within the cell.

In various examples, the cell may be exposed to the transfection material prior to and/or concurrently with the directed flow of the cell through the constriction region. In some examples, the cell is exposed after the directed flow of the cell through the constriction region, such as by using a micromixer to mix the cell with the transfection material. In some examples, the method 200 may include ejecting the cell with the apertures from the microfluidic channel to a chamber that includes the transfection material, and in response, exposing the cell with the apertures to the transfection region. The chamber may include a chamber of the microfluidic device and/or may include a chamber of a substrate external to the microfluidic device.

In some examples, the method 200 includes ejecting the transfected cell from the microfluidic channel to substrate, such as a well of a multi-well plate, using an ejection nozzle in fluidic communication with the microfluidic channel. For example, the transfected cell may be ejected from the microfluidic device to the substrate. In some examples, the ejection nozzle may form part of the fluidic pump used to direct the flow of the cell. In some examples, the ejection nozzle includes a different fluidic pump from the fluidic pump used to direct the flow of the cell.

In some examples, the microfluidic channel includes a plurality of channels. For example, the microfluidic channel may include a first channel including the constriction region and a second channel including a second constriction region. Directing the flow of the cell through the constriction region may include directing the flow of the cell to the first channel that includes the constriction region. In such examples, the method 200 may further include forming apertures in the cell membrane of a second cell of the biologic sample by directing a flow of the second cell through the second constriction region of the second channel using the fluidic pump, wherein the second constriction region includes a second circumference that is attenuated from remaining portions of the microfluidic channel and wherein the first constriction region and the second constriction region include different dimensions from one another. The method 200 may further include transfecting the second cell by exposing the second cell with the apertures to the transfection material.

As further described herein, in examples including a plurality of channels having constriction regions, the fluidic pump may include an ejection nozzle including a resistor that is in fluidic communication with the plurality of channels. In some examples, the fluidic pump may include a plurality of ejection nozzles each including a resistor, and each being in fluidic communication with a respective channel of the plurality. In some examples, the fluidic pump may include a plurality of resistors, with different resistors of the plurality being disposed within the channels or within branching channels fluidically coupled to the channels. In some examples, various combinations of ejection nozzles and resistors disposed within the channels and/or branching channels may be used. These and various additional variations are further illustrated herein.

In some examples, the method 200 may include identifying and/or optimizing the set shear force for a particular type of cell and/or for different types of cells by performing a set of optimization tests that include applying different transfection conditions on the cell type using the microfluidic channel and/or a plurality of microfluidic channels having constriction regions with different dimensions, and measuring the resulting transfection efficiency associated with the cells. Example transfection conditions include flow rate, velocity, shear force, acceleration force, deceleration force, wall friction, and shear force exposure time, among other conditions, such as force imparted on the cell by the ejection nozzle when ejecting the cell.

For example, the amount of shear force may be adjusted by changing the flow rate in a particular microfluidic channel for a plurality of cells of a particular cell type, and/or by using multiple microfluidic channels having constriction regions with different dimensions and/or by changing the flow rate in the multiple microfluidic channels. The flow rate may be changed, for example, by adjusting the velocity of the flow using the fluidic pump, such as by activating a resistor with different pulse widths, pulse frequencies, and/or for a different amount of time, and/or activating different amounts of resistors to impart different amounts of momentum to the fluid and thereby change the fluid velocity. The flow rate, which may be referred to as a volume flow rate, may be defined as:

Flow rate=velocity×cross-sectional area of the microfluidic channel,

where the cross-sectional area is defined by the circumference and/or diameter of the microfluidic channel. The flow rate and amount of constriction may impact the compression, tensile, and/or shear forces applied on the cell. For example, by adjusting the activation of fluidic pump and/or using a constriction region with an adjusted circumference, the flow rate may change and which causes different amounts of shear force to be applied on respective cells, with the adjusted circumference further causing a different compression force applied on respective cells. In some examples, a change in the length of the constriction region may cause application of the shear force for a different amount of time. In some examples, the length, shape, and taper of the circumference transitions between constriction regions and remaining portions of the microfluidic device may be used to adjust the flow rate changes and the associated amount of compression, tensile, and/or shear forces on the cell membrane which may impact the formation and size of the apertures. For example, the taper of the circumference transitions between respective constriction regions and respective remaining portions of the microfluidic device may contribute and/or cause compression, tensile, and/or shear forces due to acceleration and/or deceleration forces applied as the respective cells enter and exit the constriction regions. A plurality of cells may be tested by exposing the cells to the different compression, tensile, and/or shear forces using the apparatus, and analyzing the resulting transfection efficiency of the cells. The process may be repeated for a plurality of different cell types.

In some examples, a list of set shear force for the different types of cells may be generated from the optimization tests and stored by the apparatus, such as the apparatus 110 of FIG. 1A. For example, circuitry coupled to the fluidic pump may store the list, which identifies the set shear force for the different types of cells and/or the set flow rate used to cause the set shear force. In some examples, in which the apparatus includes a plurality of microfluidic channels, the list may identify different flow rates for different constriction regions which may be used to apply the set shear force. In some examples, the apparatus may include the stored list, and may further optimize the set shear forces over time using feedback data.

FIG. 3 illustrates another example apparatus including a first reservoir, a microfluidic channel, a second microfluidic channel, and a fluidic pump, in accordance with the present disclosure.

The first reservoir 312 may store a biologic sample containing a plurality of cells 322-1, 322-2, 322-3, 322-4, 322-5, 322-6, 322-7. In some examples, the first reservoir 312 may further store transfection material, as previously described. In other examples, the transfection material is stored separately in a second reservoir.

The first microfluidic channel 314-1 is fluidically coupled to the first reservoir 312. The first microfluidic channel 314-1 includes a first constriction region 320-1 that includes a first circumference 321-1 that is attenuated from remaining portions of the first microfluidic channel 314-1.

The second microfluidic channel 314-2 is fluidically coupled to the first reservoir 312. The second microfluidic channel 314-2 includes a second constriction region 320-2 that includes a second circumference 321-2 that is attenuated from remaining portions of the second microfluidic channel 314-2.

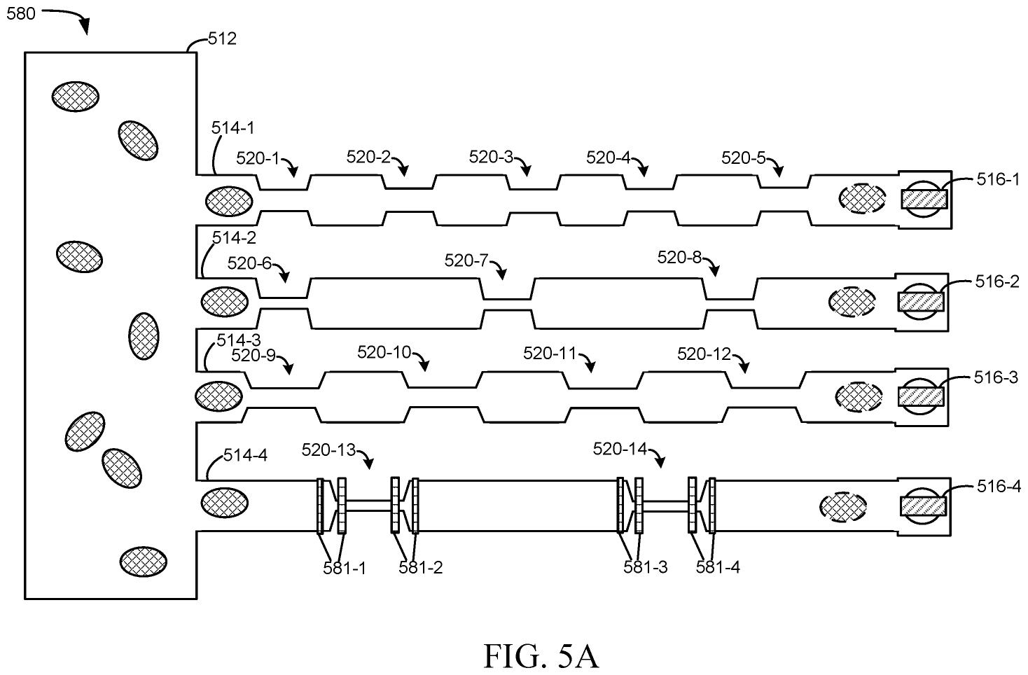

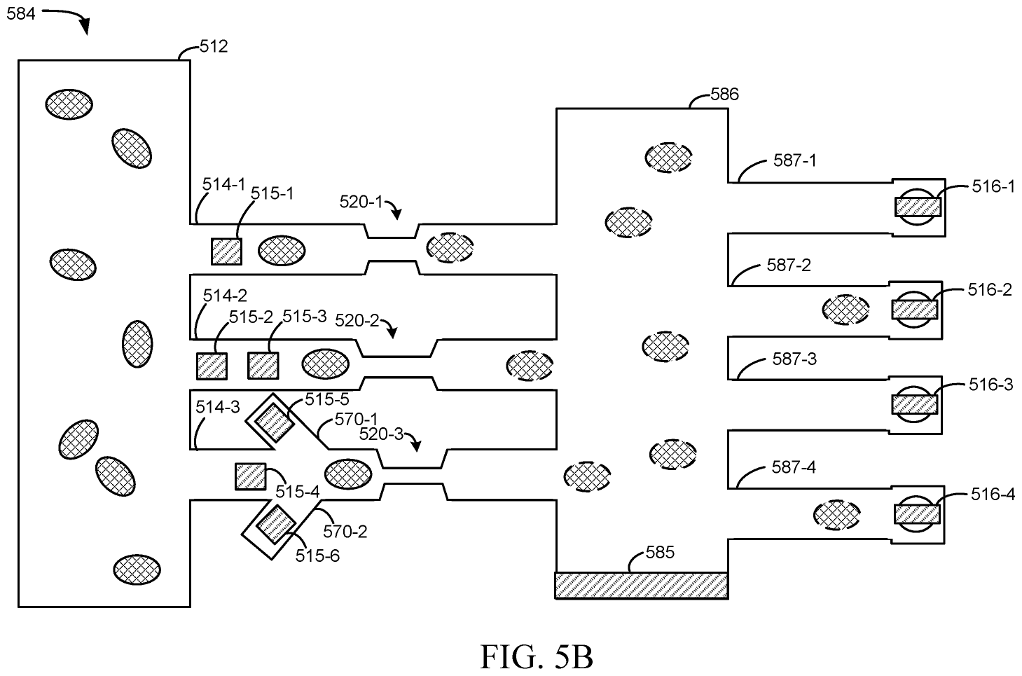

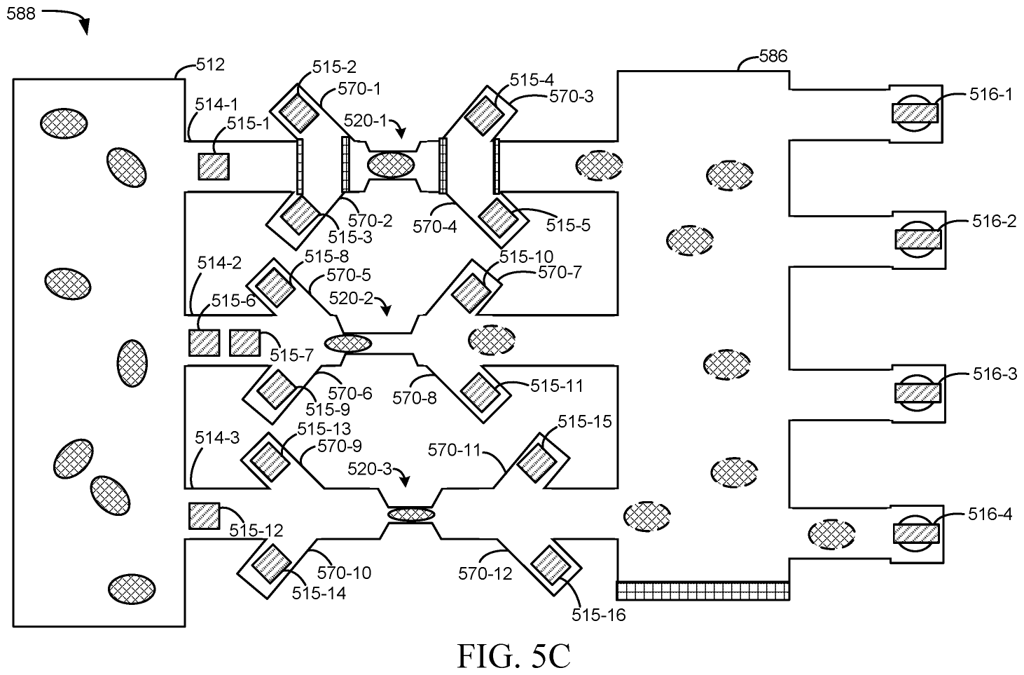

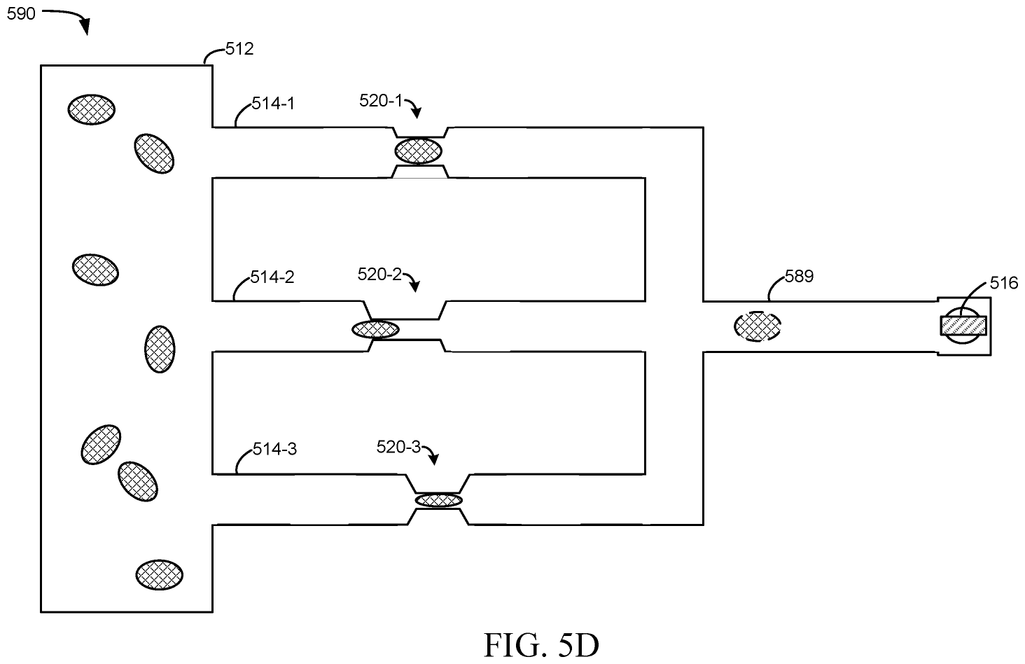

The first constriction region 320-1 and second constriction region 320-2 include different dimensions from one another. In some examples, the different dimensions of the first constriction region 320-1 and second constriction region 320-2 include circumferences, lengths, and/or number of constriction sub-regions. For example, the first constriction region 320-1 and second constriction region 320-2 may have different circumferences from one another. In some examples, the first constriction region 320-1 and second constriction region 320-2 may have different lengths and different circumferences from one another. In some examples, the first constriction region 320-1 and second constriction region 320-2 may have different numbers of constriction sub-regions, such as the first constriction region 320-1 having five constriction sub-regions and the second constriction region 320-2 having three constriction sub-regions, such as further illustrated by FIG. 5A.

The different dimensions may be used to provide different amounts of shear forces on respective cells of the plurality of cells. In some examples, additional forces, such as different tensile and/or compression forces are applied on the cells, as previously described. For example, the first constriction region 320-1 includes the first circumference 321-1 and a first length 326-1, and the second constriction region 320-2 includes the second circumference 321-2 and a second length 326-2. The second circumference 321-2 is smaller than the first circumference 321-1, and the second and first lengths 326-1, 326-2 are the same, in the particular example illustrated by FIG. 3. However, examples are not so limited. In some examples, the circumferences 321-1, 321-2 may be the same and the lengths 326-1, 326-2 are different. In some examples, both the circumferences 321-1, 321-2 and the lengths 326-1, 326-2 are different. In some examples, the apparatus 330 may include more than two microfluidic channels with constriction regions, each having different dimensions. In some examples, the constriction regions 320-1, 320-2 may have different circumferences in a range of four to ten micrometer (μm) wide and different lengths in the range of eight to thirty μm long. The circumferences may be adjusted, for example, by changing the width or height of the respective microfluidic channels. In some examples, each of the microfluidic channels 314-1, 314-2 may include multiple constriction sub-regions, as further described herein.

The fluidic pump is in fluidic communication with the first microfluidic channel 314-1 and the second microfluidic channel 314-2 to cause movement of respective portions of the biologic sample from the first reservoir 312 to the first microfluidic channel 314-1 and to the second microfluidic channel 314-2 and respectively through the first constriction region 320-1 and the second constriction region 320-2. Various fluidic pumps may be used to actively move fluid through the first and second microfluidic channel 314-1, 314-2 (and other microfluidic channels). The fluidic pump may form part of the microfluidic device, and are controlled by circuitry 318, as further described herein.

Fluid flowing through each microfluidic channel 314-1, 314-2 may be induced by operation of a single fluidic pump or multiple fluidic pumps located within the microfluidic channels 314-1, 314-2. In some examples, the microfluidic channels 314-1, 314-2 include an array of resistors which form the fluidic pump or a plurality of fluidic pumps, such as an array resistors located inside and along the length of the microfluidic channels 314-1, 314-2, which may be individually and/or collectively activated to pump fluid, to transfect cells, and/or to eject fluid from the microfluidic channels 314-1, 314-2. The fluidic pump(s) may act as a push pump by pushing fluid and/or pull pumps by pulling fluid.

While fluidic pumps may move the biologic sample through the microfluidic channels 314-1, 314-2 in the form of a fluid flow, examples are not so limited. For example, the fluidic pumps may move the biologic sample by agitating the fluid within the microfluidic channels 314-1, 314-2 and without creating a fluid flow. The fluidic pump may be individually actuated to induce a different respective flow of fluid within the microfluidic channels 314-1, 314-2.

In some examples, the fluidic pump may include a first resistor disposed with the first microfluidic channel 314-1 and a second resistor disposed with the second microfluidic channel 314-2, such as the resistors 316-1, 316-2 of the first ejection nozzle and the second ejection nozzle. For example, the resistors 316-1, 316-2 of the first ejection nozzle and second ejection nozzle may be activated to pull fluid along the first microfluidic channel 314-1 and the second microfluidic channel 314-2. In some examples and/or in addition, the fluidic pump may include the resistor 315-1 disposed within the first microfluidic channel 314-1 and the resistor 315-2 disposed within the second microfluidic channel 314-2. The resistors 315-1, 315-2 may be activated to pull and/or push fluid along the first microfluidic channel 314-1 and the second microfluidic channel 314-2, such as pulling fluid from the first reservoir 312 into the first microfluidic channel 314-1 and the second microfluidic channel 314-2, and pushing fluid along the first microfluidic channel 314-1 and the second microfluidic channel 314-2.

In some examples, the apparatus 330 further includes circuitry 318 that may be used to control components of the apparatus 330, such as the circuitry 118 previously described in connection with FIG. 2. For example, the apparatus 330 may include a microfluidic device and the circuitry 318 controls components of the microfluidic device. The microfluidic device may include the first reservoir 312, the first and second microfluidic channels 314-1, 314-2, and the fluidic pump. In some examples, the circuitry 318 forms part of the microfluidic device. The circuitry 318 may activate the fluidic pump to direct a flow of portions of the biologic sample from the first reservoir 312 into the first microfluidic channel 314-1 and through the first constriction region 320-1, and from the first reservoir 312 into the second microfluidic channel 314-2 and through the second constriction region 320-2 at different flow rates. In some examples, the different flow rates may be due to the different circumferences 321-1, 321-2 of the constriction regions 320-1, 320-2. In other examples and/or in addition, the different flow rates are due to activating the first resistor 316-1 and second resistor 316-2 differently (e.g., for different amounts of time) or activating different amounts of resistors respectively coupled to the first microfluidic channel 314-1 and second microfluidic channel 314-2. In some examples, the circuitry 318 may store a list that identifies the set flow rate for the particular type of cell and which is different for the constriction regions 320-1, 320-2 and/or may generate the list, as previously described.

As an example, the circuitry 318 may be communicatively coupled to the first resistor 316-1 and the second resistor 316-2 to activate the fluidic pump to direct a flow of a first portion of the biologic sample from the first reservoir 312 into the first microfluidic channel 314-1 and through the first constriction region 320-1, and to direct a flow of a second portion of the biologic sample from the first reservoir 312 into the second microfluidic channel 314-2 and through the second constriction region 320-2. The first portion of the biologic sample may include a first cell 322-4 of the plurality of cells and the second portion may include a second cell 322-5 of the plurality of cells. In some examples, the circuitry 318 may activate the fluidic pump to apply a first shear force on the first cell 322-4 in response to the flow through the first constriction region 320-1, and thereby cause formation of apertures in the cell membrane of the first cell 322-4. The circuitry 318 may activate the fluidic pump to apply a second shear force on the second cell 322-5 in response to the flow through the second constriction region 320-2, and thereby cause formation of apertures in the cell membrane of the second cell 322-5. In some examples, the circuitry 318 may activate the fluidic pump to apply sets of forces. For example, the circuitry 318 may apply a first set of compression, tensile, and shear forces, including the first shear force, on the first cell 322-4. The circuitry 318 may apply a second set of compression, tensile, and shear forces, including the second shear force, on the second cell 322-5. The circuitry 318 may further activate the fluidic pump to expose the first cell 322-4 and the second cell 322-5 to transfection material to transfect the first cell 322-4 and second cell 322-5.

As described above, the different dimensions of the first constriction region 320-1 and second constriction region 320-2 may include a dimension selected from circumference, length, number of constriction sub-regions, and combinations thereof. In some examples, the circuitry 318 is to activate the fluidic pump to apply a first shear force on the first cell 322-4 in response to the flow through the first constriction region 320-1, and thereby cause formation of apertures in the cell membrane of the first cell 322-4, apply a second shear force on the second cell 322-5 in response to the flow through the second constriction region 320-2, and thereby cause formation of apertures in the cell membrane of the second cell 322-5, and expose the first cell 322-4 and second cell 322-5 to transfection material to transfect the first cell 322-4 and the second cell 322-5. For example, the first resistor 316-1 may be activated to cause the flow of fluid through the first microfluidic channel 314-1 and the second resistor 316-2 may be activated to cause the flow of fluid through the second microfluidic channel 314-2, and which may occur simultaneously or at different times.

The apparatus 110 illustrated by FIGS. 1A-1B and the apparatus 330 illustrated by FIG. 3 may include a variety of variations, including but not limited to different types of fluidic pumps, different placement and/or number of resistors used to form the fluidic pump(s), sensors, and different arrangement of microfluidic channels. As a further example, the apparatus 330 may include additional microfluidic channels than illustrated by FIG. 3, and as further illustrated herein.

FIGS. 4A-4H illustrate a variety of different example apparatuses for transfecting cells, in accordance with the present disclosure. The apparatuses 440, 446, 450, 456, 462, 468, 474, 478 may include substantially the same features as previously described by FIG. 1A. For example, each of the apparatuses 440, 446, 450, 456, 462, 468, 474, 478 include a first reservoir 412, a microfluidic channel 414, a constriction region 420, and a fluidic pump. Each apparatus 440, 446, 450, 456, 462, 468, 474, 478 may include a microfluidic device and coupled circuitry used to control components of the microfluidic device, such as activating a fluidic pump that is an integrated component of the microfluidic device. In some examples, each of the apparatuses 440, 446, 450, 456, 462, 468, 474, 478 include an ejection nozzle with a resistor 416 which may be activated by the circuitry to eject the cell from the microfluidic channel 414. The various features illustrated by FIGS. 4A-4H may be used in different combinations in accordance with examples disclosed herein.

In some examples, the apparatuses 440, 446, 450, 456, 462, 468, 474, 478 may include fluidic pumps formed by a plurality of resistors and/or that may be considered multiple fluidic pumps. As shown by FIG. 4A, an example apparatus 440 includes a fluidic pump formed by a plurality of resistors 442. The plurality of resistors 442 may include different sized and/or strength resistors which are co-located within the microfluidic channel 414. In the example, the plurality of resistors 442 may be located on an end of the microfluidic channel 414 that is opposite from the first reservoir 412. The plurality of resistors 442 may be selectively activated by circuitry to generate different backpressure in the first reservoir 412 and to adjust the flow rate of fluid flowing through the microfluidic channel 414. The circuitry may activate each resistor individually and/or in different combinations to generate different flow rates.

As shown by FIG. 4B, an example apparatus 446 may include a fluidic pump formed by a plurality of resistors 415-1, 415-2, 415-3 that are disposed within the microfluidic channel 414. The plurality of resistors 415-1, 415-2, 415-3 may be in series and may be upstream from the constriction region 420. The plurality of resistors 415-1, 415-2, 415-3 may push fluid to direct the flow of the cell into and through the constriction region 420. In some examples, circuitry may synchronously activate the plurality of resistors 415-1, 415-2, 415-3 to increase the flow rate through the constriction region 420. The circuitry may activate each resistor individually and/or in different combinations to generate different flow rates.

As shown by FIG. 4C, an example apparatus 450 may include a second reservoir 452 that is fluidically coupled to the microfluidic channel 414. The second reservoir 452 may store the transfection material. In the particular example, the apparatus 450 includes a fluidic pump formed by a plurality of resistors 442, as previously described by FIG. 4A. However, examples are not so limited and different fluidic pumps may be used in the apparatus 450, such as those illustrated by FIGS. 4B and 4D-4H. In some examples, circuitry may activate the fluidic pump formed by the plurality of resistors 442 to drive the flow of the cell and transfection material from the first reservoir 412 and the second reservoir 452 into the microfluidic channel 414 and through the constriction region 420.

As shown by FIG. 4D, an example apparatus 456 may include a fluidic pump formed by a plurality of resistors 415-1, 415-2, 415-3 that are disposed within the microfluidic channel 414. The plurality of resistors 415-1, 415-2, 415-3 may be in series and may be downstream from the constriction region 420. The plurality of resistors 415-1, 415-2, 415-3 may pull fluid to direct the flow of the cell into and through the constriction region 420. In some examples, circuitry may synchronously activate the plurality of resistors 415-1, 415-2, 415-3 to increase the flow rate through the constriction region 420. The circuitry may activate each resistor individually and/or in different combinations to generate different flow rates.

As another example, the apparatus 462 of FIG. 4E includes a first resistor 415-1 disposed within the microfluidic channel that is upstream from the constriction region 420 and a second resistor 415-2 that is downstream from the constriction region 420. The first resistor 415-1 may be activated to push fluid in a first direction (e.g., to the right), thereby pushing the fluid through the constriction region 420. The second resistor 415-2 may be activated to push the fluid in a second direction (e.g., to the left) that is opposite the first direction and pushes the fluid back through the constriction region 420. Circuitry may selectively activate the first resistor 415-1 and the second resistor 415-2 to direct the cell back and forth through the constriction region 420 a plurality of times and to provide a set amount of shear force on the cell, among other forces.

In some examples, the resistors forming the fluidic pumps may be located in channels that intersect or are branched from the microfluidic channel that the cell travels through. By locating the resistors in a separate channel, effects from the resistors on the cell may be mitigated and/or removed. As shown by FIG. 4F, an example apparatus 468 may include a fluidic pump formed by a first resistor 415-1 and a second resistor 415-2, a microfluidic channel 414, and two branching channels 470-1, 470-2 that intersect the microfluidic channel 414. The first resistor 415-1 is disposed within the first branching channel 470-1 and the second resistor 415-2 is disposed within the second branching channel 470-2. The circuitry may activate the first and second resistors 415-1, 415-2 to direct the flow within the microfluidic channel 414.

As shown by FIG. 4G, an example apparatus 474 may include a fluidic pump formed by a plurality of resistors 416-1, 416-2, 416-3, 416-4, 416-5. The plurality of resistors 416-1, 416-2, 416-3, 416-4, 416-5 may each form part of an ejection nozzle, and are selectively activated to control the flow rate. For example, the greater number of resistors 416-1, 416-2, 416-3, 416-4, 416-5 that are activated, the greater the flow rate. The circuitry may activate one, two, three, four, or five of the resistors 416-1, 416-2, 416-3, 416-4, 416-5 at the same time to vary the flow rate and, therefore, vary the shear force applied on cells. In some examples, the plurality of ejections nozzles may be fluidically coupled to the microfluidic channel 414 by a channel arrangement 476 which includes first channel that intersects the microfluidic channel 414 and a plurality of branching channels in fluidic communication with the first channel and in which the ejection nozzles are respectively disposed within. While the example of FIG. 4G illustrates five resistors, examples are not so limited and may include more or less resistors.

Various examples may include different combinations of features of the above-described apparatus, such as different combinations of resistors and ejection nozzles used to form a fluidic pump. As shown by FIG. 4H, an example apparatus 478 may include a fluidic pump formed by a first resistor 415-1 disposed within a first branching channel 470-1, a second resistor 415-2 disposed within a second branching channel 470-2, a third resistor 415-3 disposed within the microfluidic channel 414, and a plurality of resistors 416-1, 416-2, 416-3, 416-4, 416-5 that each form part of an ejection nozzle and may be fluidically coupled to the microfluidic channel 414 by a channel arrangement 476, as previously described herein. Circuitry may selectively activate different ones of the resistors 415-1, 415-2, 415-3, 416-1, 416-2, 416-3, 416-4, 416-5 to set the flow rate.

FIGS. 5A-5D illustrate a variety of different example apparatuses for transfecting cells and which include multiple microfluidic channels, in accordance with the present disclosure. The apparatuses 580, 584, 588, 590 may include substantially the same features as previously described by FIG. 3. For example, each of the apparatuses 580, 584, 588, 590 include a first reservoir 512, a first microfluidic channel 514-1, a second microfluidic channel 514-2, and a fluidic pump. Examples are not limited to two microfluidic channels, and may include a third microfluidic channel 514-3, a fourth microfluidic channel 514-4 or more microfluidic channels that each include a constriction region with different dimensions from one another. In some examples, the constriction regions may have varied circumferences, such as being four to ten μm wide, and/or have varied lengths, such as being eight to thirty μm long. Each apparatus 580, 584, 588, 590 may include a microfluidic device and coupled circuitry used to control components of the microfluidic device, such as activating the fluidic pump that is an integrated component of the microfluidic device. The features illustrated by FIGS. 5A-5D may be used in different combinations in various examples.

In some examples, each of the plurality of microfluidic channels may include a plurality of constriction regions, sometimes herein referred to as “constriction sub-regions”. Each of the plurality of microfluidic channels may have two to five constriction regions, with the dimensions of the constriction regions varying between the different channels. As shown by FIG. 5A, an example apparatus 580 may include four microfluidic channels 514-1, 514-2, 514-3, 514-4, 514-5. The first microfluidic channel 514-1 includes five constriction regions 520-1, 520-2, 520-3, 520-4, 520-5 which have the same dimensions. The second microfluidic channel 514-2 includes three constriction regions 520-6, 520-7, 520-8 which have the same dimensions. The third microfluidic channel 514-3 includes four constriction regions 520-9, 520-10, 520-11, 520-12 which have the same dimensions. The fourth microfluidic channel 514-4 includes two constriction regions 520-13, 520-14. The dimensions of the constriction regions 520-1, 520-2, 520-3, 520-4, 520-5 of the first microfluidic channel 514-1 are different from the remaining, e.g., 520-6, 520-7, 520-8, 520-9, 520-10, 520-11, 520-12, 520-13, 520-14.

In the particular example, ejection nozzles that include resistors 516-1, 516-2, 516-3, 516-4 are disposed within each of the microfluidic channels 514-1, 514-2, 514-3, 514-4, and may be activated by coupled circuitry to drive the flow of cells through each of the microfluidic channels 514-1, 514-2, 514-3, 514-4, as previously described. However, examples are not so limited and other arrangements of resistors may be used to form a fluidic pump and to direct the flow of cells through the microfluidic channels 514-1, 514-2, 514-3, 514-4. The additional constriction regions with the microfluidic channels 514-1, 514-2, 514-3, 514-4 may increase the amount of time that the cells are exposed to compression, tensile, and/or shear forces, thereby increasing the amount of force(s) applied on the cells.

In some examples, sensors may be located within the microfluidic device and used to detect the presence of a cell. As an example, the fourth microfluidic channel 514-4 of the apparatus 580 includes a first sensor 581-1 and a second sensor 581-2 located proximal to the first constriction region 520-13, and a third sensor 581-3 and a fourth sensor 581-4 located proximal to the second constriction region 520-14. The sensors may include sets of electrodes which are used to detect a sensor signal that indicates whether a cell is present. In some examples, circuitry is coupled to the sensors and used to count the number of cells which may be transfected. In some examples, the sensor signals may be used by the circuitry to activate the fluidic pump, such as to transfect one cell at a time per channel. In some examples, in response to the sensor signal indicating the presence of a cell, the circuitry may activate respective resistors 516-1, 516-2, 516-3, 516-4 to eject cells from the microfluidic channel 514-1, 514-2, 514-3, 514-4.

In some examples, the sensor signals may be used to tune the set flow rates for a type of cell. For example, the apparatus 580 may be used to optimize flow rates and/or other transfection conditions for the type of cell, as previously described.

Various apparatuses with multiple microfluidic channels may have different arrangements of resistors used to direct the flow of fluid within the apparatus. As shown by FIG. 5B, an example apparatus 584 may include three microfluidic channels 514-1, 514-2, 514-3 and a second reservoir 586 located between the microfluidic channels 514-1, 514-2, 514-3 and the ejection nozzles including the resistors 516-1, 516-2, 516-3, 516-4. The first microfluidic channel 514-1 includes a resistor 515-1 disposed within the first microfluidic channel 514-1, which is activated to drive cells through the first constriction region 520-1 and to the second reservoir 586. The second microfluidic channel 514-2 includes two resistors 515-2, 515-3 disposed within the second microfluidic channel 514-2, which may be activated to drive cells through the second constriction region 520-2 and to the second reservoir 586. The third microfluidic channel 514-3 includes a resistor 515-4 disposed within the third microfluidic channel 514-3 and two resistors 515-5, 515-6 disposed within branching channels 570-1, 570-2 which may be activated to drive cells through the third constriction region 520-3 and to the second reservoir 586. Circuitry may selectively activate different ones of the resistors and/or activate the respective resistors differently to provide different flow rates and/or to provide the set flow rate through each of the microfluidic channels 514-1, 514-2, 514-3.

As previously described, the second reservoir 586 may allow for the cells to incubate with the transfected material and/or to recover from the shear force and to aggregate. In some examples, the second reservoir 586 may include a potential mixer 585 to mix the cells with transfection material.

In the illustrated example, the ejection nozzles include resistors 516-1, 516-2, 516-3, 516-4 disposed within each of additional channels 587-1, 587-2, 587-3, 587-4 that are fluidically coupled to the second reservoir 586. The ejection nozzles may be used to eject the transfected cells, as previously described. However, examples are not so limited and in some examples the transfected cells may be extracted in other way, such as by pipetting.

Other example combinations of resistors may be used to provide for different tuneability of the forces applied and the flow rates. As shown by FIG. 5C, an example apparatus 588 includes three microfluidic channels 514-1, 514-2, 514-3, the second reservoir 586 located between the microfluidic channels 514-1, 514-2, 514-3, and the ejection nozzles including the resistors 516-1, 516-2, 516-3, 516-4, similar to apparatus 584 but with different combinations of resistors. In the particular example, the first microfluidic channel 514-1 includes a resistor 515-1 disposed within the first microfluidic channel 514-1, two resistors 515-2, 515-3 disposed within branching channels 570-1, 570-2 upstream of the first constriction region 520-1, and two resistors 515-4, 515-5 disposed within branching channels 570-3, 570-4 downstream from the first constriction region. Similarly, the second and third microfluidic channels 514-2, 514-3 include resistors 515-6, 515-7, 515-12 disposed within the second and third microfluidic channels 514-2, 514-3, two resistors 515-8, 515-9, 515-13, 515-14 disposed within branching channels 570-5, 570-6, 570-9, 570-10 upstream of the constriction regions 520-2, 520-3, and two resistors 515-10, 515-11, 515-15, 515-16 disposed within branching channels 570-7, 570-8, 570-11, 570-12 downstream from the constriction regions 520-2, 520-3. The resistors may be selectively activated to drive cells back and forth through the constriction regions 520-1, 520-2, 520-3 and to the second reservoir 586.

In some examples, the microfluidic channels 514-1, 514-2, 514-3 may include sensors proximal to the constriction regions 520-1, 520-2, 520-3, as previously described. The sensors may be a variety of different types of sensors used to provide monitoring and/or feedback to the circuitry. Example sensors include an impedance sensor, an image sensor, a light sensor, among other types of sensors. For an impedance sensor, the buffer solution may be non-conductive, such as a phosphate buffer. As cells are conductive, an impedance detected by the impedance sensor may indicate the presence of a cell. In some examples, previously detected cells may subsequently not provide a sensor signal, which may indicate that the cell is lysed and used to optimize the set flow rates, as further described herein. For the light sensor, light scattering with the microfluidic channels may be monitored. The light sensor may be used with bacteria and small cells, which may be difficult to sense using an image sensor.

In some examples, a single resistor or single fluidic pump may be used to drive the flow of fluid through multiple channels. As shown by FIG. 5D, an example apparatus 590 includes an ejection nozzle including the resistor 516 which is fluidically coupled to three microfluidic channels 514-1, 514-2, 514-3 via the additional channel 589. The resistor 516 may be activated by circuitry to drive the flow of cells from the first reservoir 512 through the microfluidic channels 514-1, 514-2, 514-3 including the constriction regions 520-1, 520-2, 520-3. In some examples, the flow rate provided by the apparatus 590 may be slower than an apparatus with multiple resistors.

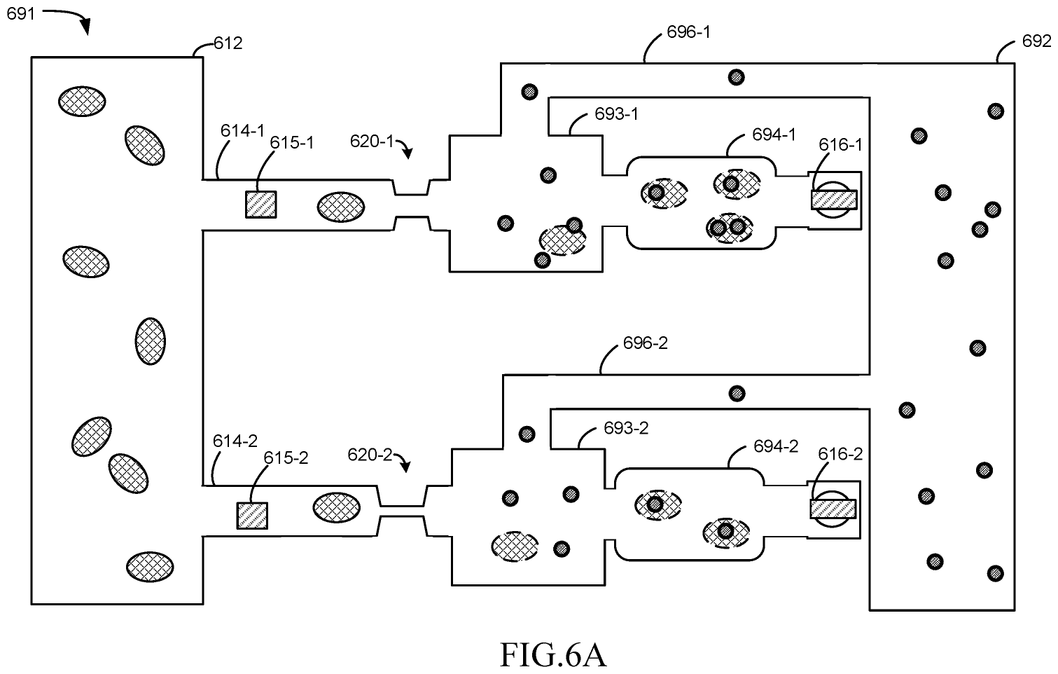

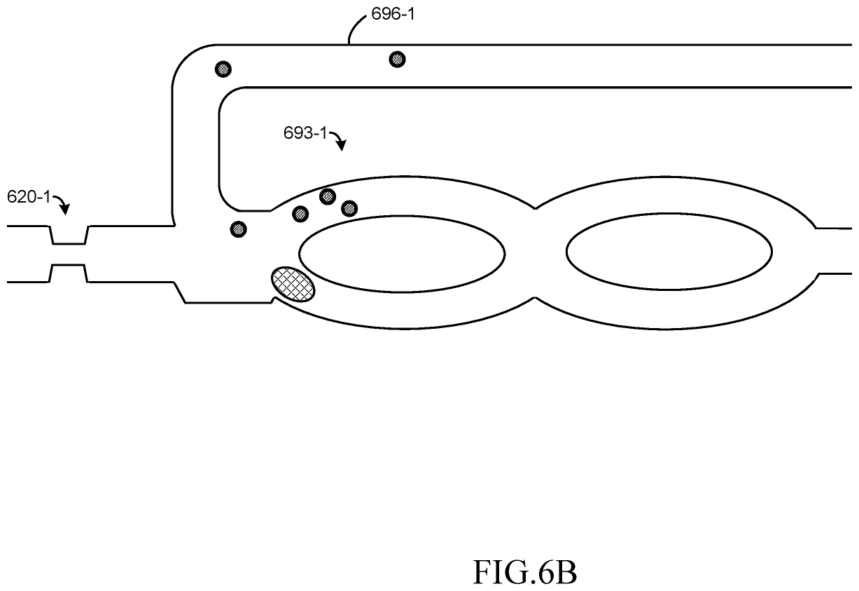

FIGS. 6A-6B illustrate an example apparatus for transfecting cells and which include a micromixer, in accordance with the present disclosure. As shown by FIG. 6A, the example apparatus 691 may include substantially the same features as previously described by FIG. 3. For example, the apparatus 691 includes a first reservoir 612, a first microfluidic channel 614-1, a second microfluidic channel 614-2, and a fluidic pump. In the particular example, the fluidic pump includes a first fluidic pump formed by a first resistor 615-1 disposed within the first microfluidic channel 614-1 and a second resistor 615-2 disposed within the second microfluidic channel 614-2, however examples are not so limited and may include different arrangements of resistors used to set the flow rate within the first microfluidic channel 614-1 and second microfluidic channel 614-2.

The apparatus 691 further includes a second reservoir 692 to store transfection material and a second fluidic pump fluidically coupled to the second reservoir 692. In the particular example, the second fluidic pump may be formed by the resistors 616-1, 616-2 of the ejection nozzles.

The apparatus 691 further includes a micromixer 693-1, 693-2 fluidically coupled to the second reservoir 692 and the microfluidic channels 614-1, 614-2. For example, channels 696-1, 696-2 may fluidically couple the second reservoir 692 and the micromixer 693-1, 693-2. The micromixer 693-1, 693-2 may include an arrangement of channels and/or a chamber which is used to mix cells with the transfection material. In some examples, coupled circuitry is to activate the first fluidic pump to direct the flow of cells from the first reservoir 692 into the microfluidic channels 614-1, 614-2, through the constriction regions 620-1, 620-2, and to the micromixers 693-1, 693-2. The circuitry further activates the second fluidic pump to direct a flow of the transfection material from the second reservoir 692 to micromixers 693-1, and 693-2, and to mix the cells with apertures with the transfection material to transfect the cells. The apparatus 691 may include third reservoirs 694-1, 694-2 coupled between the micromixers 693-1, and 693-2 and the ejection nozzles, which may allow for the cells to aggregate and/or incubate.

FIG. 6B illustrates a particular example of a micromixer 693-1 which includes channel in a figure-eight arrangement. For example, the micromixer 693-1 of FIG. 6B may include the micromixer 693-1 of FIG. 6A which is fluidically coupled to the first microfluidic channel 614-1, the second reservoir 692, and the particular third reservoir 694-1. The example micromixer 693-1 may include an asymmetric structure comprising the channel in the figure-eight arrangement and which may cause different flow rates of fluids. The figure-eight arrangement may cause unbalanced splits and cross-collisions of the fluid containing the cell with fluid containing the transfection material. Such an example micromixer 693-1 may be referred to as an unbalanced collision-based micromixer. However, examples are not so limited and may include different shaped asymmetric structures, spiral-based micromixers, lamination-based micromixers, chamber-based micromixers, such those including convergence-divergence structures or barriers, overbridge micromixers, and/or micromixers with an active energy source to drive the flow and mixture of fluids.

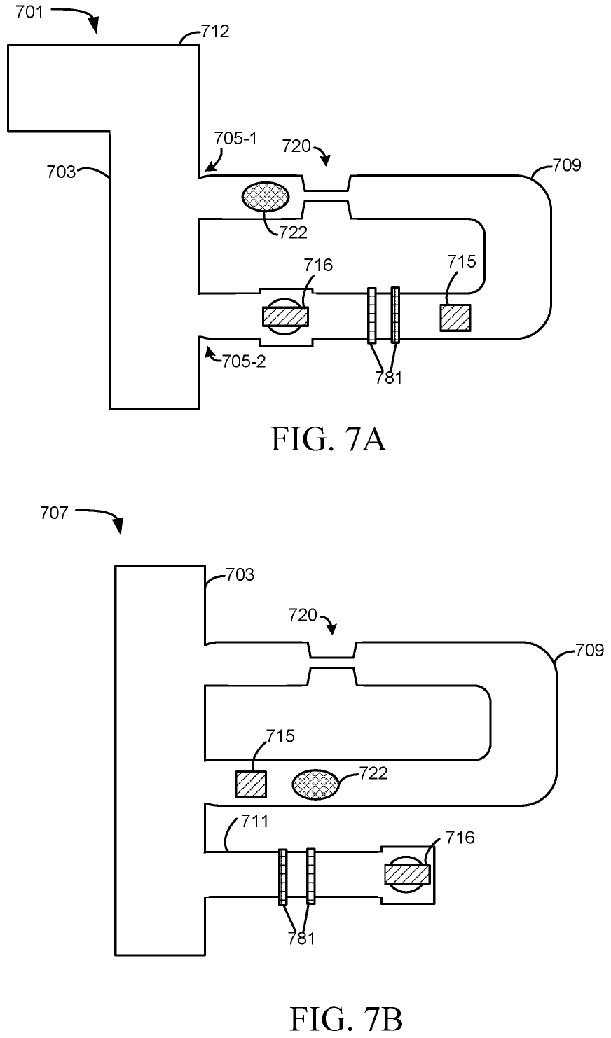

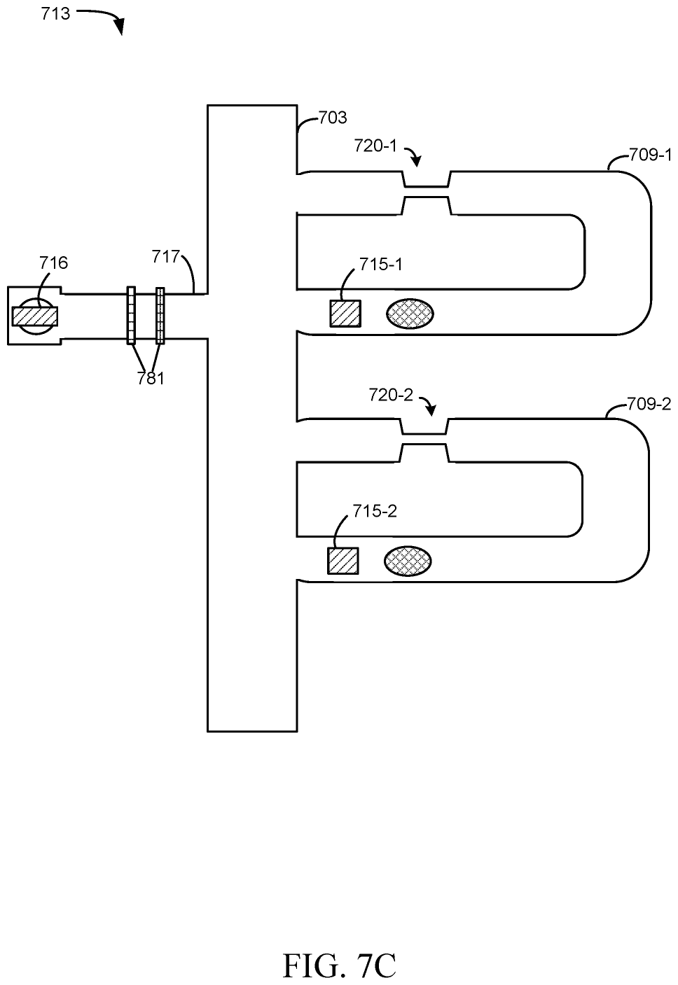

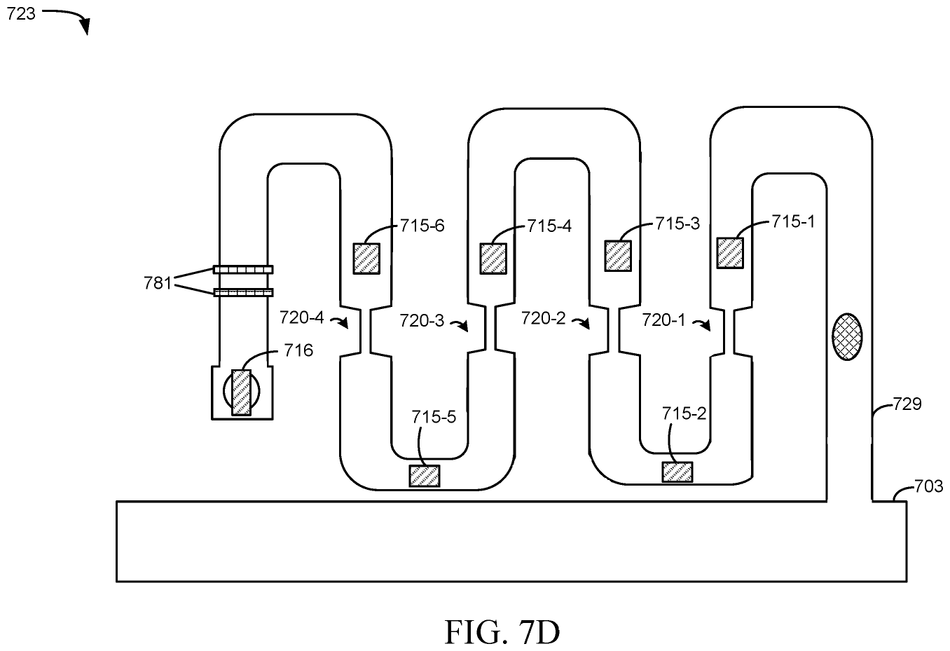

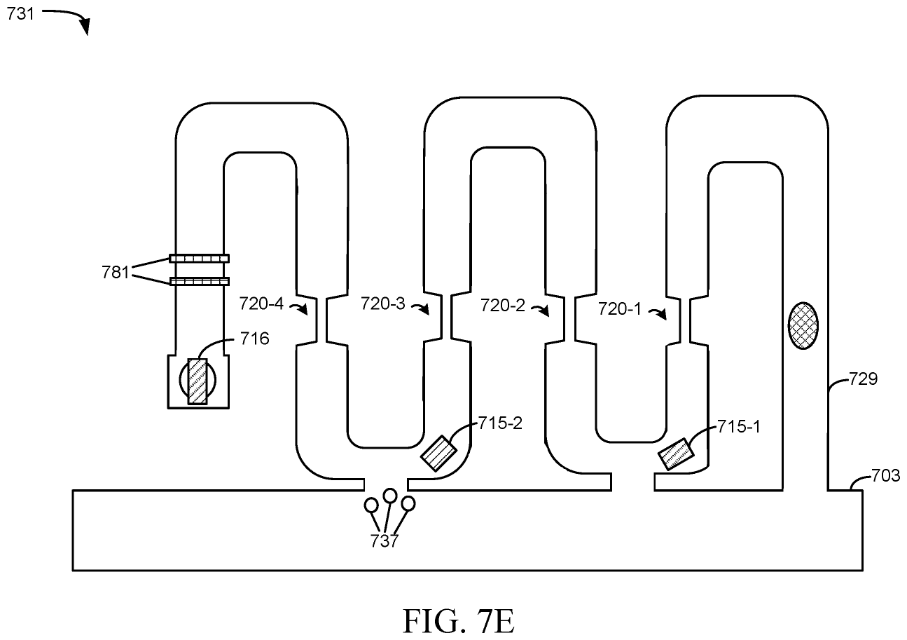

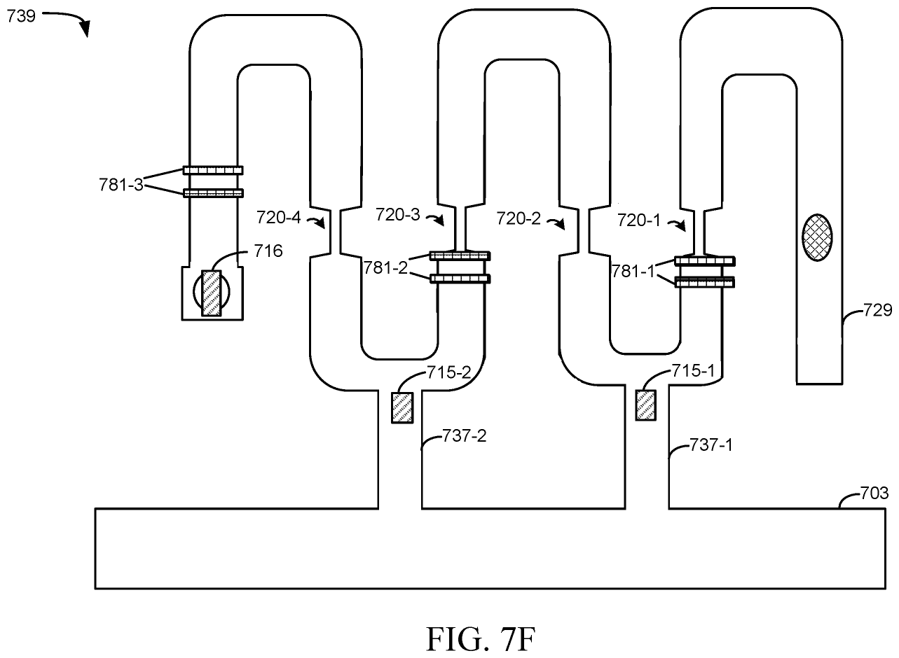

FIGS. 7A-7F illustrate a variety of different example apparatuses for transfecting cells and which include recirculation loops, in accordance with the present disclosure. The apparatuses 701, 707, 713, 723, 739 may include substantially the same features as previously described by FIG. 1A and/or FIG. 3, with the microfluidic channel and/or first and second microfluidic channels being formed in a recirculation loop 709, 709-1, 709-2. For example, each of the apparatuses 701, 707, 713, 723, 739 include a first reservoir 712 and a microfluidic channel or first and second microfluidic channels that include constriction regions 720, 720-1, 720-2, and a fluidic pump. Each of the apparatuses 701, 707, 713, 723, 739 may include a microfluidic device and coupled circuitry used to control components of the microfluidic device, such as activating the fluidic pump. The features illustrated by FIGS. 7A-7F may be used in different combinations in various examples.

As shown by FIG. 7A, an example apparatus 701 includes a microfluidic channel formed as a recirculation loop 709. A recirculation loop 709 includes a first end 705-1 and a second end 705-2 coupled to the first reservoir 712 via an intermediate channel 703. The cell 722 may recirculate along the recirculation loop 709 and shear force and/or other forces may be applied to the cell while traveling through the constriction region 720 at each recirculation. In the particular example, the fluidic pump may be formed by the resistor 715 located within the recirculation loop 709. For example, the cell 722 may be pulled into the recirculation loop 709 by the resistor 715, flowed through the recirculation loop 709, and then pushed out of and pulled back into the recirculation loop 709 to be subjected to additional shear force, among other forces. The recirculation loop 709 may include a sensor 781 and an ejection nozzle with a resistor 716. The sensor may be located upstream from the ejection nozzle in the recirculation loop 709 and the sensor signal may be used to identify when the cell is present for activating the resistor 715 to drive the flow of the cell 722 through the recirculation loop 709 and/or for activating the resistor 716 to eject the cell 722 from the recirculation loop 709.

In some examples, the ejection nozzle may be located in separate channel from the recirculation loop. As shown by FIG. 7B, an example apparatus 707 includes a microfluidic channel formed as a recirculation loop 709 and a second channel 711 that includes the sensor 781 and the ejection nozzle with the resistor 716 to eject the cell 722. Similar to the apparatus 701, the cell 722 may be pulled into the recirculation loop 709 by the resistor 715, flowed through the recirculation loop 709, and pushed out of and pulled back into the recirculation loop 709 to be subjected to additional shear and/or other forces. The resistor 716 may be used to pull the cell into the second channel 711 from the intermediate channel 703 and eject the cell from the apparatus 701.

Various examples may include multiple recirculation loops and/or a second channel at different locations of the apparatus. For example, FIG. 7C shows an example apparatus 713 that includes a first recirculation loop 709-1, a second recirculation loop 709-2, and a second channel 717 that includes the sensor 781 and the ejection nozzle with the resistor 716 to eject cells. The second channel 717 is on an opposite side of the intermediate channel 703 from the first recirculation loop 709-1 and the second recirculation loop 709-2. However, examples are not limited and example apparatuses may include multiple recirculation loops 709-1, 709-2 with an ejection nozzle in a separate channel on the same side of the intermediate channel 703 as the recirculation loops 709-1, 709-2. In some examples, recirculation loops 709-1, 709-2 may be on both sides of the intermediate channel 703.

In some examples, the microfluidic channel may include a continuous recirculation loop, such as a channel having multiple curves, as shown by FIGS. 7D-7F. As shown by FIG. 7D, an example apparatus 723 includes a microfluidic channel formed as the continuous recirculation loop 729 that is fluidically coupled to an intermediate channel 703 which may be fluidically coupled to a first reservoir 712 as shown by FIG. 7A. However, examples are not so limited and the continuous recirculation loop 729 may be directly coupled to the first reservoir. The continuous recirculation loop 729 includes a fluidic pump or a plurality of fluidic pumps formed by resistors 715-1, 715-2, 715-3, 715-4, 715-5, 715-6 disposed within the continuous recirculation loop 729. While FIG. 7D illustrates six resistors, examples are not so limited and may include more or less resistors, such as including resistors 715-2, 715-5 to drive the flow of fluid through the continuous recirculation loop 729. The continuous recirculation loop 729 includes multiple constriction regions 720-1, 720-2, 720-3, 720-4, which expose the cell to multiple applications of shear and/or other forces as the cell is flowed along the continuous recirculation loop 729. In some examples, the resistors 715-1, 715-2, 715-3, 715-4, 715-5, 715-6 may be used to drive the flow of fluid and to unclog the continuous recirculation loop 729. For example, a cell may be clogged in one of the constriction regions 720-1, 720-2, 720-3, 720-4 and respective resistors 715-1, 715-2, 715-3, 715-4, 715-5, 715-6 proximal to the clog may push and/or pull fluid back and forth to unclog the constriction region.

The continuous recirculation loop 729 may include a sensor 781 and an ejection nozzle with a resistor 716 at the end of the continuous recirculation loop 729. The sensor 781 may be located upstream from the ejection nozzle in the continuous recirculation loop 729 and the sensor signal may be used to identify when the cell is present for activating the resistor 716 to eject the cell 722 from the continuous recirculation loop 729.

FIG. 7E illustrates another example apparatus 731 with a continuous recirculation loop 729, but with fluid flow provided between an intermediate channel 703 coupled to a first reservoir (such as the first reservoir 712 illustrated by FIG. 7A) and within the continuous recirculation loop 729 by two resistors 715-1, 715-2 disposed within the continuous recirculation loop 729 and/or the resistor 716 of the ejection nozzle. For example, the continuous recirculation loop 729 and intermediate channel 703 (or the first reservoir) may have multiple fluidic couplings, which may be used to provide greater control over the fluid flow within the continuous recirculation loop 729. In some examples, the apparatus 731 further includes a filter 737, such as the illustrated pillars, at the fluidic couplings to prevent the cell flowing along the continuous recirculation loop 729 from re-entering the intermediate channel 703 and/or to prevent other cells or particles from entering the continuous recirculation loop 729 other than at the intended entry point for the continuous recirculation loop 729. Although not illustrated, in some examples, the first reservoir may be directly coupled to the continuous recirculation loop 729.

The filter 737 may include a plurality of pillars that are spaced to prevent cells or other particles from flowing through. The pillars may be a variety of different shapes, such as elongated pillars and/or different geometric shaped pillars. In other examples, the filter 737 may include a solid structure with apertures formed therein, with the apertures providing fluidic communication between the sides of the filter 737. The filter 737 may be formed of a variety of materials, such as SU8 material, epoxy-based negative photoresist material, and/or other suitable material.

Similar to the apparatus 731 of FIG. 7E, FIG. 7F illustrates an example apparatus 739 with a continuous recirculation loop 729, but with the flow being directed by two resistors 715-1, 715-2 disposed within branching channels 737-1, 737-2 that are fluidically coupled to the continuous recirculation loop 729 and the intermediate channel 703 or first reservoir. Although not illustrated, the branching channels 737-1, 737-2 may include filters, as previously described.

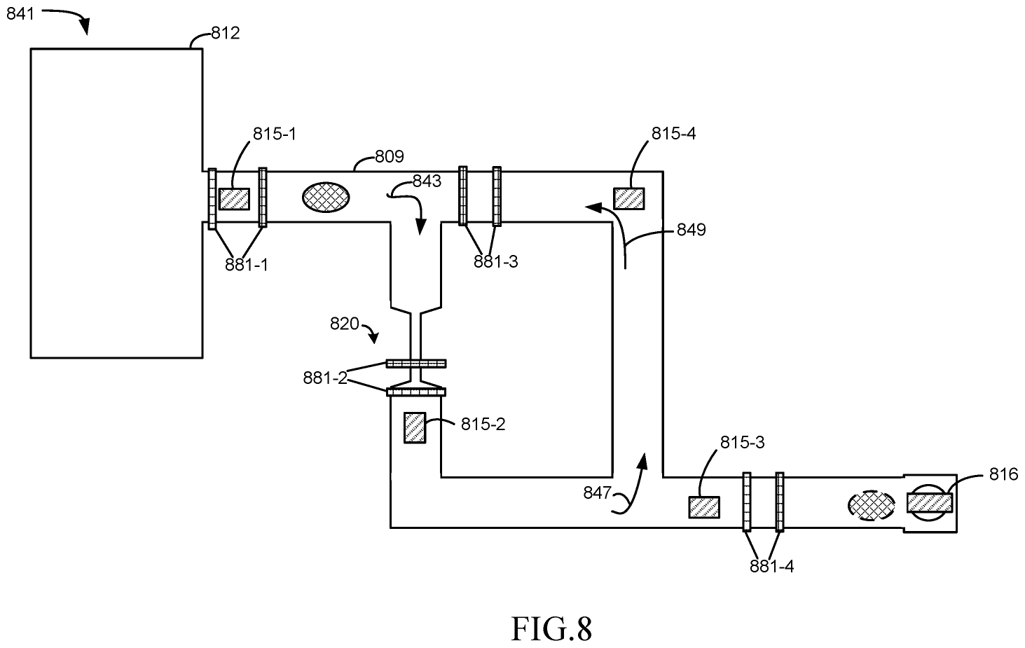

FIG. 8 Illustrates an example apparatus for performing cell transfection using a recirculation loop, in accordance with the present disclosure. The apparatus 841 may include substantially the same features as previously described by FIG. 2A, FIG. 3 and/or FIG. 7A, with the microfluidic channel and/or first and second microfluidic channels being formed in a recirculation loop 809. For example, the apparatus 841 includes a first reservoir 712 and a recirculation loop 809 that include a constriction region 820, and a fluidic pump. The apparatus 841 may include a microfluidic device and coupled circuitry used to control components of the microfluidic device, such as activating the fluidic pump that is an integrated component of the microfluidic device. Although the recirculation loop 809 is illustrated with a single constriction region 820, examples are not so limited and may include multiple constriction regions.

As shown, the fluidic pump is formed by a plurality of resistors 815-1, 815-2, 815-3, 815-4, 816 that are disposed within the recirculation loop 809 and used to drive the flow of the cell and/or fluid through the recirculation loop 809. The recirculation loop 809 may optionally include a plurality of sensors 881-1, 881-2, 881-3, 881-4 which may be used to detect the presence of the cell. In some examples, the sensor signals are used to track a number of times a particular cell has travelled through the constriction region 820 and/or a number of cells that have been transfected. In some examples, the sensor signals are used by coupled circuitry to determine when to activate the plurality of resistors 815-1, 815-2, 815-3, 815-4, 816 to drive the flow of the cell through the recirculation loop 809 and/or to eject the cell.

Circuitry may be coupled to the plurality of resistors 815-1, 815-2, 815-3, 815-4, 816 to selectively activate the plurality of resistors 815-1, 815-2, 815-3, 815-4, 816 at different times and/or differently to drive the flow of fluid and to apply a set shear force to the cell. For example, the circuitry may simultaneously activate the second resistor 815-2 and resistor 816 of the ejection nozzle to direct the flow of a cell from the first reservoir 812 to the recirculation loop 809 and through the constriction region 820, as shown by the arrow 843. The circuitry may simultaneously activate the second resistor 815-2 and the third resistor 815-3 to drive the cell back into the recirculation loop 809, as shown by the arrow 847. The circuitry may simultaneously activate the first resistor 815-1 and the fourth resistor 815-4 to drive the cell back toward the constriction region 820, as shown by the arrow 849. The circuitry may then simultaneously activate the second resistor 815-2 and resistor 816 of the ejection nozzle to direct the flow of the cell through the constriction region 820 again. The process may be repeated, in some examples, until a set shear force (and/or other forces) has been applied to the cell, and the circuitry may activate the resistor 816 to eject the cell from the apparatus 841.

The various figures herein illustrate particular numbers of microfluidic channels, constriction regions, recirculation loops, and resistors. However, examples are not limited, and may include variety of different orientations. Although the various apparatus illustrate symmetrical designs, examples are not so limited. For example, the microfluidic devices may include high through-put and/or parallel designs.

Circuitry as used herein, such as circuitry 118 and 318, include a processor, machine readable instructions, and other electronics for communicating with and controlling the fluidic pumps, and other components of the apparatus, such as the sensor, the fluidic pump(s) and/or resistor(s), and other components. The circuitry may receive data from a host system, such as a computer, and includes memory for temporarily storing data. The data may be sent to the apparatus along an electronic, infrared, optical, or other information transfer path. A processor may be a central processing unit (CPU), a semiconductor-based microprocessor, a graphics processing unit (GPU), a microcontroller, special purpose logic hardware controlled by microcode or other hardware devices suitable for retrieval and/or execution of instructions stored in a memory, or combinations thereof. In addition to or alternatively to retrieving and executing instructions, the processor may include at least one integrated circuit (IC), other control logic, other electronic circuits, or combinations thereof that include a number of electronic components for performing the function. In some examples, the circuitry includes non-transitory computer-readable storage medium that is encoded with a series of executable instructions that may be executed by the processor. Non-transitory computer-readable storage medium may be an electronic, magnetic, optical, or other physical storage device that contains or stores executable instructions. Thus, non-transitory computer-readable storage medium may be, for example, Random Access Memory (RAM), an Electrically Erasable Programmable Read-Only Memory (EEPROM), a storage device, an optical disc, etc. In some examples, the computer-readable storage medium may be a non-transitory storage medium, where the term ‘non-transitory’ does not encompass transitory propagating signals.

A biologic sample, as used herein, refers to any biological material, collected from a subject. Examples of biologic samples include, but are not limited to, whole blood, blood plasma, and other body fluids, as well as tissue cell cultures obtained from humans, plants, or animals. Such biologic samples may contain any viral or cellular material, including all prokaryotic or eukaryotic cells, viruses, bacteriophages, mycoplasmas, protoplasts, and organelles. The biological material may comprise all types of mammalian and non-mammalian animal cells, plant cells, algae including blue-green algae, fungi, bacteria, protozoa, etc. Non-limiting sample examples include whole blood and blood-derived products such as plasma, serum and buffy coat, urine, feces, cerebrospinal fluid or other body fluids, tissues, cell cultures, cell suspensions, etc. The term “fluid”, as used herein, refers to any substance that flows under applied shear stress. In some examples, the fluid includes the biologic sample including an analyte and/or a reagent or reactant, among others.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. An apparatus, comprising:
a first reservoir to store a biologic sample containing a cell;
a microfluidic channel fluidically coupled to the first reservoir, the microfluidic channel including:
a constriction region including a first circumference that is attenuated from remaining portions of the microfluidic channel; and
a fluidic pump disposed within the microfluidic channel; and
circuitry to activate the fluidic pump to direct flow of the cell from the first reservoir to the microfluidic channel and through the constriction region.

2. The apparatus of claim 1, wherein the circuitry is to activate the fluidic pump to direct the flow of the cell to:
apply shear force on the cell in response to the flow through the constriction region, and thereby cause formation of apertures in the cell membrane of the cell; and
expose the cell to transfection material to transfect the cell.

3. The apparatus of claim 1, wherein the fluidic pump includes a plurality of resistors disposed within the microfluidic channel, and the circuitry is to selectively activate the plurality of resistors to provide a first flow rate to flow the cell to the microfluidic channel and provide a second flow rate to flow the cell into and through the constriction region, wherein the second flow rate includes a change in velocity from the first flow rate.

4. The apparatus of claim 1, further including:
a second reservoir fluidically coupled to the microfluidic channel, the second reservoir to store transfection material; and
wherein the circuitry is to activate the fluidic pump to:
direct the flow of the cell from the first reservoir to the microfluidic channel and through the constriction region and direct flow of the transfection material from the second reservoir to the microfluidic channel and through the constriction region to transfect the cell.

5. The apparatus of claim 1, wherein:
the microfluidic channel includes a first channel including the constriction region and a second channel that intersects and is in fluidic communication with the first channel, the fluidic pump being disposed within the second channel.

6. The apparatus of claim 1, further including an ejection nozzle disposed within the microfluidic channel, wherein the circuitry is to activate a resistor of the ejection nozzle to eject the cell from the microfluidic channel to a chamber, wherein the chamber includes transfection material, and in response, exposes the cell to the transfection material.

7. The apparatus of claim 1, wherein:
the microfluidic channel includes a first channel including the constriction region and a second channel including a second constriction region, wherein the second constriction region includes a second circumference that is attenuated from remaining portions of the microfluidic channel and wherein the first constriction region and the second constriction region include different dimensions from one another; and
the circuitry is to activate the fluidic pump to:
direct the flow of the cell from the first reservoir to the first channel and through the constriction region; and
direct flow of a second cell from the first reservoir to the second channel and through the second constriction region.

8. A method, comprising receiving, at a microfluidic channel, a biologic sample containing a cell and transfection material;
forming apertures in the cell membrane of the cell by directing a flow of the cell through a constriction region of the microfluidic channel, wherein the flow of the cell is directed using a fluidic pump disposed within the microfluidic channel, and wherein the constriction region includes a first circumference that is attenuated from remaining portions of the microfluidic channel; and
transfecting the cell by exposing the cell with the apertures to the transfection material.

9. The method of claim 8, wherein directing the flow of the cell through the constriction region includes changing a flow of a portion of the biologic sample from a first velocity to a second velocity, thereby flowing the cell into the constriction region and constricting the cell, and causing application of a set shear force on the cell.

10. The method of claim 8, wherein directing the flow of the cell through the constriction region includes:
applying compression, tensile, and shear forces on the cell by directing the flow of the cell into and through the constriction region; and
removing the compression, tensile, and shear forces on the cell by directing the flow of the cell out of the constriction region, and the method further including:
incubating the cell with the transfection material to allow the transfection material to pass through the apertures and the apertures to close, thereby trapping the transfection material in the cell.

11. The method of claim 8, further include ejecting the transfected cell from the microfluidic channel to a substrate using a nozzle in fluidic communication with the microfluidic channel.

12. An apparatus, comprising:
a first reservoir to store a biologic sample containing a plurality of cells;
a first microfluidic channel fluidically coupled to the first reservoir, the first microfluidic channel including a first constriction region that includes a first circumference that is attenuated from remaining portions of the first microfluidic channel;
a second microfluidic channel fluidically coupled to the first reservoir, the second microfluidic channel including a second constriction region that includes a second circumference that is attenuated from remaining portions of the second microfluidic channel, the first constriction region and second constriction region including different dimensions from one another; and
a fluidic pump in fluidic communication with the first microfluidic channel and the second microfluidic channel to actuate and cause movement of respective portions of the biologic sample from the first reservoir to the first microfluidic channel and to the second microfluidic channel and respectively through the first constriction region and the second constriction region.

13. The apparatus of claim 12, wherein the fluidic pump includes:
a first resistor disposed with the first microfluidic channel;
a second resistor disposed with the second microfluidic channel; and
circuitry communicatively coupled to the first resistor and the second resistor to activate the fluidic pump to:
direct a flow of a first portion of the biologic sample from the first reservoir into the first microfluidic channel and through the first constriction region, the first portion including a first cell of the plurality of cells; and
direct flow a flow of a second portion of the biologic sample from the first reservoir into the second microfluidic channel and through the second constriction region, the second portion including a second cell of the plurality of cells.

14. The apparatus of claim 13, wherein the different dimensions of the first constriction region and second constriction region include a dimension selected from circumference, length, number of constriction sub-regions, and combinations thereof, and the circuitry is to activate the fluidic pump to:
apply a first shear force on the first cell in response to the flow through the first constriction region, and thereby cause formation of apertures in the cell membrane of the first cell;
apply a second shear force on the second cell in response to the flow through the second constriction region, and thereby cause formation of apertures in the cell membrane of the second cell; and
expose the first cell and second cell to transfection material to transfect the first cell and the second cell.

15. The apparatus of claim 12, further including circuitry to activate the fluidic pump and direct a flow of the portions of the biologic sample from the first reservoir into the first microfluidic channel and through the first constriction region, and from the first reservoir into the second microfluidic channel and through the second constriction region at different flow rates.
